(19) United States
(12) Patent Application Publication
(10) Pub. No.: US 2005/0113680 A1
(43) Pub. Date: May 26, 2005

(54) Cerebral ischemia diagnosis assisting apparatus, X-ray computer tomography apparatus, and apparatus for aiding diagnosis and treatment of acute cerebral infarct

(76) Inventors: Yoshihiro Ikeda, Nasu-gun (JP); Kieran Murphy, Baltimore, MD (US); Miwa Okumura, Kuroiso-shi (JP); Yasuko Fujisawa, Otawara-shi (JP); Yuusuke Toki, Tokyo (JP); Yasuhiro Noshi, Nasu-gun (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/971,010

(22) Filed: Oct. 25, 2004

(30) Foreign Application Priority Data
Oct. 29, 2003 (JP) 2003-368893
Oct. 29, 2003 (JP) 2003-368894

(51) Int. Cl. A61B 5/05

(52) U.S. Cl. 600/425; 128/920; 378/4

(57) ABSTRACT

A cerebral ischemia diagnosis assisting apparatus comprises a storing portion for storing multi-slices or volume data with regard to the head portion of a subject, an image generating portion for generating a tomography image of a brain from the multi-slices or the volume data, an image processing portion for processing the tomography image for generating a contrast highlighting image and a brain sulci highlighting image or either thereof, and a display portion for displaying the tomography image along with the contrast highlighting image and the brain sulci highlighting image.

### CROSS-REFERENCE TO RELATED APPLICATIONS

This application is based upon and claims the benefit of priority from prior Japanese Patent Applications No. 2003-368893, filed Oct. 29, 2003; and No. 2003-368894, filed Oct. 29, 2003, the entire contents of both of which are incorporated herein by reference.

### BACKGROUND OF THE INVENTION

1. Field of the Invention

The present invention relates to a cerebral ischemia diagnosis assisting apparatus, an X-ray computer tomography apparatus equipped with a function thereof, and an apparatus for aiding the diagnosis and treatment of acute cerebral infarct by offering information useful for diagnosis and determination of treatment policy of acute cerebral infarction.

2. Description of the Related Art

It is very important in the medical field to qualitatively determine a position and a size of an ischemia region and a degree of seriousness thereof by reading an X-ray CT image of the brain in acute cerebral infarction and a degree of seriousness thereof and swiftly determine applicability of treatment at an initial stage of diagnosis. In order to determine the ischemia region and the degree, it is necessary to read a portion bringing about a reduction in a CT value or a portion at which the cerebral sulci disappears from the CT image.

However, actually, it is a current state that the reduction in the CT value or the disappearance of the cerebral sulci caused by the ischemia is very delicate and cannot be read without a medical specialist rich in experience and knowledge.

Further, in the medical field, high accuracy is required, as well as speed, in diagnosing cerebral infarction in the acute phase and determining the treatment policy. For example, when an ischemic region is found, and if a treatment for dissolving the blood clot is immediately made, there is a danger of bleeding. Therefore, meticulous attention is necessary. A rich amount of knowledge and experience is required for the diagnosis of cerebral infarction in the acute phase and for the determination of the treatment policy.

### BRIEF SUMMARY OF THE INVENTION

It is an object of the invention to assist to read a cerebral ischemia region from a simple (nonconstructed) CT image by a user.

It is an other object of the present invention to provide information useful for the diagnosis of acute cerebral infarction and for the determination of the treatment policy.

According to a first aspect of the invention, there is provided a cerebral ischemia diagnosis assisting apparatus comprising a storing portion for storing multi-slices or volume data with regard to the head portion of a subject, an image generating portion for generating a tomography image of a brain from the multi-slices or the volume data, an image processing portion for processing the tomography image for generating a contrast highlighting image and a cerebral sulci highlighting image or either thereof, and a display portion for displaying the tomography image along with the contrast highlighting image and the cerebral sulci highlighting image.

According to a second aspect of the invention, there is provided an apparatus for aiding the diagnosis and treatment of acute cerebral infarction comprising a storage portion for storing non-contrast enhanced CT images regarding cross sections of the head of a patient to be examined and cerebral blood flow images representing the spatial distribution of indexes about the dynamic state of the cerebral blood flow regarding cross sections of the patient substantially identical to the aforementioned cross sections, an image processing portion for superimposing a first ROI (region of interest) about ischemia identified from the CT images and a second ROI about low blood flow identified from the cerebral blood images onto the CT images, and a display portion for displaying the CT images on which the first and second ROIs have been superimposed. According to the invention, the two kinds of ROIs (i.e., ischemic region and low blood flow region) are superimposed on the CT images and displayed. This is useful in diagnosing acute cerebral infarction and determining the treatment policy.

According to a third aspect of the invention, there is provided an apparatus for aiding the diagnosis and treatment of acute cerebral infarction, comprising a storage portion for storing a three-dimensional CT image of the head of a patient and a three-dimensional contrast CT image of the head of the patient, an image processing portion for superimposing a three-dimensional image of a first ROI in the infract region identified from the CT image and a three-dimensional image of a second ROI in a low-blood flow region identified from the contrast CT image onto a blood vessel image generated from the contrast CT image, and a display portion for displaying the image of the blood vessels, which is superimposed with the three-dimensional images of the first and second ROIs.

Additional objects and advantages of the invention will be set forth in the description which follows, and in part will be obvious from the description, or may be earned by practice of the invention. The objects nd advantages of the invention may be realized and obtained by means of the instrumentalities and combinations particularly pointed out herein after.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is a diagram showing an outline constitution of an X-ray computer tomography apparatus according to a first embodiment of the invention;

FIG. 2 is a flowchart of diagnosis assisting operation according to the first embodiment;

FIG. 3 is a detailed flowchart of the diagnosis assisting operation according to the first embodiment;

FIG. 4 is a view exemplifying a center line of the brain for supplementing an image display direction correcting processing of FIG. 3;

FIG. 5 is a view exemplifying a central face A for separating the left and right cerebral hemispheres prescribed from the center line of FIG. 4 for supplementing the image display direction correcting processing of FIG. 3);

FIG. 6 is a view showing coordinates correction of a volume data based on the central face for supplementing the image display direction correcting processing of FIG. 3;

FIG. 7 is a view showing a method of manually designating the central face of FIG. 5;

FIG. 8 is an explanatory view of a method of automatically detecting the center line of FIG. 4;

FIG. 9 is a view exemplifying an interpolating processing for generating a left and right inverted image used in the method of automatically detecting the center line of FIG. 4;

FIG. 10 is a view showing an example of a display screen according to the embodiment;

FIG. 11 is a view showing an example of a display screen according to the embodiment;

FIG. 12 is a detailed view of a guide line of FIG. 11;

FIG. 13 is a diagram schematically showing the structure of an X-ray CT scanner fitted with an apparatus for aiding the diagnosis and treatment of acute cerebral infarction, the apparatus being according to a second embodiment of the present invention;

FIGS. 14A and 14B show examples of two kinds of images according to the second embodiment;

FIG. 15 is a flowchart illustrating a sequence of operations for aiding the diagnosis and treatment of acute cerebral infarction according to the second embodiment;

FIGS. 16A-16C are supplemental views to steps S4, S8, and S9 of the flowchart of FIG. 15;

FIG. 17 is a flowchart illustrating a sequence of operations for aiding the diagnosis of ischemia using CT images, the operations being included in the sequence of operations for aiding the diagnosis and treatment of acute cerebral infarction according to the second embodiment;

FIG. 18 is a view taken through the center line of the brain, supplementarily illustrating the processing of FIG. 17 for correcting the direction of display of image;

FIG. 19 shows the center plane A that separates the right and left cerebral hemispheres, the plane being defined from the center line of FIG. 18, supplementarily illustrating the processing of FIG. 17 for correcting the direction of display of image;

FIG. 20 is a diagram illustrating correction of coordinates indicated by volume data based on the center plane, supplementarily illustrating the processing of FIG. 17 for correcting the direction of display of image;

FIG. 21 is a diagram illustrating a method of manually specifying the center plane of FIG. 19;

FIG. 22 is a diagram illustrating a method of automatically detecting the center line of FIG. 18;

FIG. 23 is a diagram illustrating interpolation processing for creating right and left inverted images used in the method of automatically detecting the

FIG. 24 is a diagram showing an example of the display screen in step S11 of the flowchart of FIG. 17;

FIG. 25 is a diagram showing an example of the display screen in step S12 of the flowchart of FIG. 17;

FIG. 26 is a detail diagram of the guidelines of FIG. 25;

FIG. 27 is a flowchart illustrating a sequence of operations for the diagnosis and treatment of acute cerebral infarction using the apparatus according to the present aspect for aiding the diagnosis and treatment of acute cerebral infarction;

FIG. 28 is a diagram illustrating the principle of CBP study made by the CBP study processing portion of FIG. 13;

FIG. 29 is a diagram illustrating MTF processing performed by the CBP study processing portion of FIG. 13;

FIGS. 30A-30D show examples of cerebral blood flow images such as CBP maps created by the CBP study processing portion of FIG. 13;

FIG. 31 is a diagram schematically showing the structure of an X-ray CT scanner having an apparatus for aiding the diagnosis and treatment of acute cerebral infarction, the apparatus being one according to a third embodiment of the present invention;

FIG. 32 is a flowchart explaining the process of aiding the diagnosis and treatment of acute cerebral infarction, which is performed by the third embodiment;

FIG. 33 is an additional diagram for explaining the process explained by FIG. 32; and

FIG. 34 is a diagram showing a 3D-angiogram of the type shown in FIG. 33.

### DETAILED DESCRIPTION OF THE INVENTION

An explanation will be given of embodiments of the invention in reference to the drawings as follows. Further, in an X-ray computer tomography apparatus, there are various types of a rotation/rotation type for integrating an X-ray tube and a radiation detector to rotate at a surrounding of a subject, a fix/rotation type in which a number of detecting elements are arrayed in a ring-like shape and only an X-ray tube is rotated at a surrounding of a subject and so on and the invention is applicable to any of the types.

An explanation will be given here of the rotation/rotation type currently constituting the main current.

Further, in order to reconstitute a tomographic image data at one slice, there is needed a projected data of an amount of one turn or about 360 degrees at the surrounding of the subject or projected data of an amount of 180°+α (α: fan angle) also in a half scan method. The invention is applicable to either of the reconstitution systems. An explanation will be given here of the half scan method as an example. Further, according to a mechanism of converting incident X-ray into electric charge, the main current is constituted by an indirect conversion type for converting X-ray into light by a fluorescent substance of a scintillator or the like and further converting the light into an electric charge by a photoelectric conversion element of a photodiode or the like and a direct conversion type utilizing generation of electron hole pairs at inside of a semiconductor by X-ray and movement thereof to the electrodes, that is, a photoconductive phenomenon. Although either of the types may be adopted as an X-ray detecting element, an explanation will be given here of the former of the indirect conversion type. Further, in recent years, product formation of an X-ray computer tomography apparatus of a so-to-speak multiple tube type in which a plurality of pairs of X-ray tubes and X-ray detectors are mounted on a rotating ring has been progressed and development of a peripheral technology thereof has been progressed. The invention is applicable to a single tube type X-ray computer tomography apparatus of the related art or an X-ray computer tomography apparatus of a multiple tube type. An explanation will be given here of the single tube type.

### FIRST EMBODIMENT

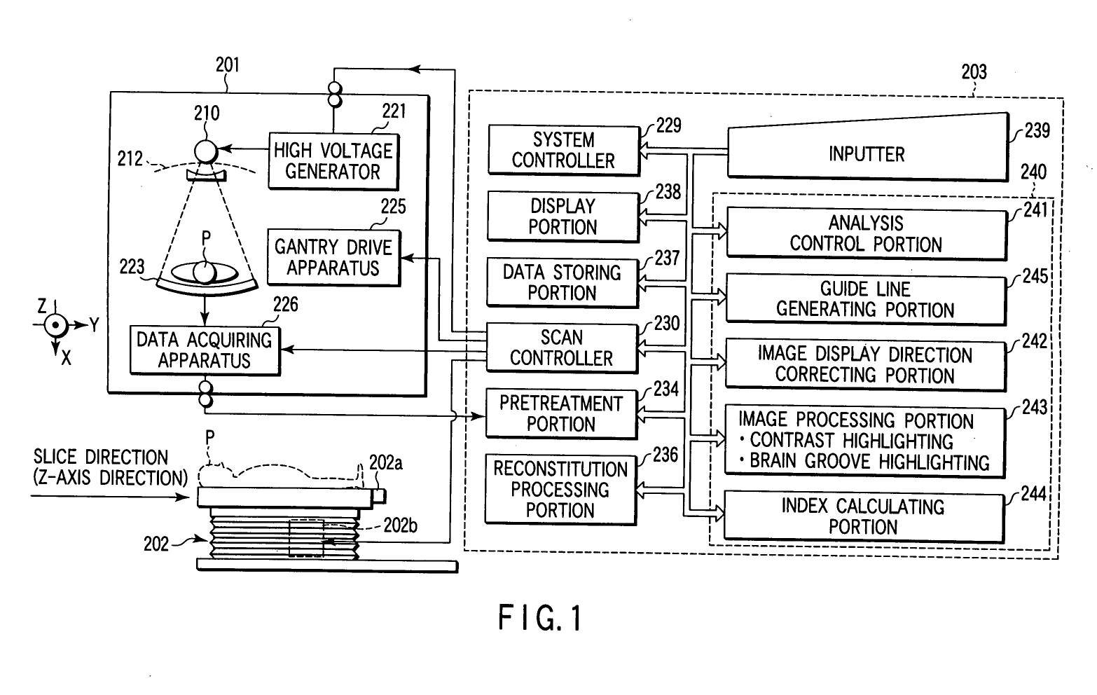

As shown by FIG. 1, an X-ray computer tomography apparatus includes a gantry 201 constituted for acquiring projected data with regard to a subject. The gantry 201 includes an X-ray tube 210 and an X-ray detector 223. The X-ray tube 210 and the X-ray detector 223 are mounted to a rotating frame 212 in a ring-like shape driven to rotate by a gantry drive apparatus 225. A central portion of the rotating frame 212 is opened and a subject P mounted on a patient couch top 202a of a patient couch 202 is inserted into the opening portion. Further, a rotational center axis of the rotating frame 212 is defined by Z-axis (slice direction axis) and a plane orthogonal to Z-axis is defined by orthogonal two axes of X and Y.

A tube voltage is applied from a high voltage generator 221 between a cathode and an anode of the X-ray tube 210 and a filament current is supplied to a filament of the X-ray tube 210 from the high voltage generator 221. X-ray is generated by applying the tube voltage and supplying the filament current. As the X-ray detector 223, either of a one-dimensional array type detector and a two-dimensional array detector (also referred to as multi-slide type detector) may be adopted. The X-ray detecting element includes a light receiving face of, for example, 0.5 mm×0.5 mm square. For example, 916 pieces of the X-ray detecting elements are aligned in a channel direction. The two-dimensional array type detector is constituted by aligning the rows in the slice direction by, for example, 40 columns. The one-dimensional alloy type detector is constituted by a single column thereof.

A data acquiring apparatus 226 generally referred to as DAS (data acquisition system) converts a signal outputted from the detector 223 for each channel into a voltage signal, amplifies the voltage signal and converts the voltage signal into a digital signal. The data (raw data) is supplied to a computer unit 203 outside of the gantry. A preprocessing portion 234 of the computer unit 203 subjects the data (raw data) outputted from the data acquiring apparatus 226 to a correction processing of sensitivity correction or the like to output a projected data. The projected data is transmitted to store to a data storing apparatus 237 of the computer system 203.

The computer system 203 is constituted by a system controller 229, a scan controller 230, a reconstitution processing portion 236, a display portion 238, an input device 239 having a mouse, a keyboard or the like and a cerebral ischemia diagnosis assisting apparatus (also referred to as cerebral ischemia analysis (CT-DE) apparatus) 240 along with the preprocessing portion 234 and the storing portion 237. The reconstitution processing portion 236 reconstitutes an image data of multi-slice or volume (hereinafter, referred to as volume data for convenience of explanation) by an arbitrarily reconstituting method of a Feldkamp reconstituting method or a computer reconstituting method based on projected data acquired by, for example, helical scan, volume scan using cone beam X-ray, or both. Further, the multi-slice data is an aggregation of a number of sheets of images expressed by an XY coordinates system Z positions of which slightly differ from each other at constant slice intervals and a slice number and generally a resolution (slice interval) in the slice direction (Z-axis) is longer than a resolution (pixel pitch) of X and Y axes. The volume data is an aggregation of voxels of cubic bodies, expressed by XYZ coordinates system. The reconstituted volume data is transmitted to store to the data storing apparatus 237.

The cerebral ischemia diagnosis assisting apparatus 240 includes a cerebral ischemia analysis control portion 241, an image display direction correcting portion 242, an image processing portion 243, an index calculating portion 244 and a guidance generating portion 245. The image display direction correcting portion 242 corrects a display direction of a tomography image initially generated from the volume data. Actually, the image display direction correcting portion 242 manually or automatically detects a central face separating the left and right cerebral hemispheres from the tomography image initially generated and generates a tomography image of a face orthogonal to the central face separating the left and right cerebral hemispheres by multiplanar reconstruction processing (MPR) from the volume data. The image processing portion 243 processes the tomography image (referred to as original image) generated by the image display direction correcting portion 242, determines presence or absence of hemorrhage and generates a contact highlighting image and a cerebral sulci highlighting image. The original image is displayed on the display portion 238 along with the contrast highlighting image and the cerebral sulci highlighting image under control of the system controller 229 or the CT-DE control portion 241. The contrast highlighting image is an image in which the brain tissue region of the original image is classified into at least two kinds of colors in accordance with CT values. The cerebral sulci highlighting image is an image in which the cerebral spinal fluid region of the original image is classified into at least two kinds of colors in accordance with CT values. The index calculating portion 244 calculates, for example, a ratio of volumes of the left cerebral hemisphere region and the right cerebral hemisphere region, a ratio between areas of the left cerebral hemisphere region and the right cerebral hemisphere region of the original image and a correlation coefficient between the original image and a left and right inverted image centering on the center line of the original image as useful indices of the cerebral ischemia diagnosis representing a symmetry between the left cerebral hemisphere and right cerebral hemisphere. The guidance generating portion 245 determines applicability of treatment (thromboloclasis method) based on a combination of a plurality of diagnosis results by determination of the user.

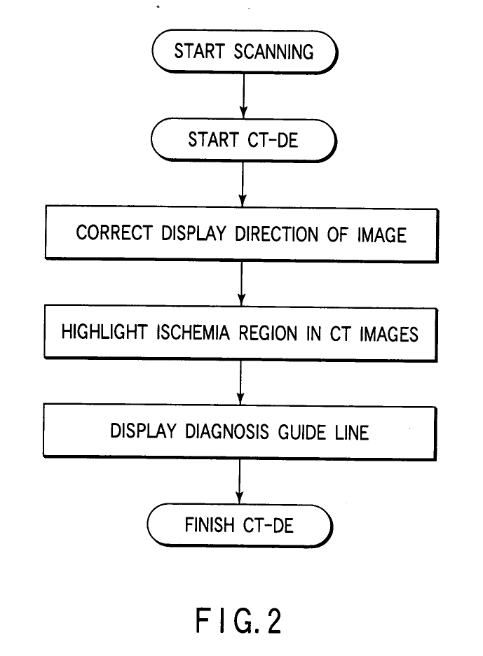

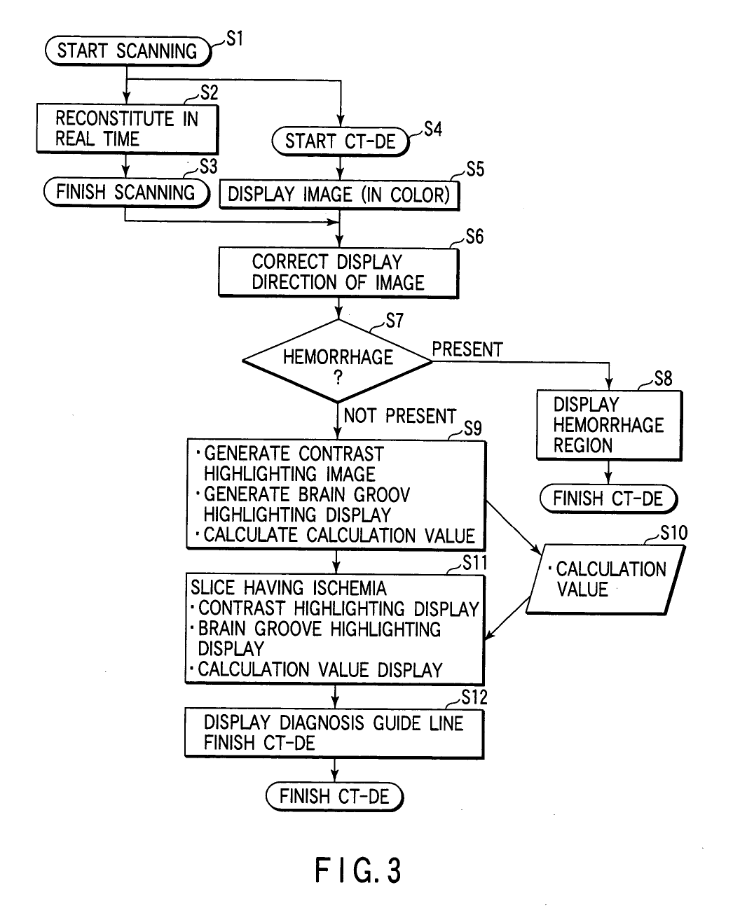

FIG. 2 shows an outline flow of a cerebral ischemia diagnosis assisting processing according to the embodiment. FIG. 3 shows a detailed flow of the cerebral ischemia diagnosis assisting processing according to the embodiment. First, scanning is started (S1). The scanning is helical scan or volume scan constituting a range thereof by the head portion of the subject. A tomography image is successively reconstituted in parallel with scanning at each time of acquiring projected data of an amount of an angle necessary for reconstituting one sheet of tomography image (S2). A technology of successively reconstituting the tomography image in parallel with the scanning is a well-known technology referred to as real time CT or CT fluoroscopy. The scanning is finished at a time point at which data of predetermined range including the head portion of the subject is finished to acquire (S3).

With start of scanning, a function for cerebral ischemia analysis (CT-DE) is started by the analysis control portion 241 (S4). The image processing portion 243 controlled by the analysis control portion 241 classifies the tomography image reconstituted by real time CT into, for example, 5 kinds of colors to facilitate discovery of the hemorrhage region (S5). The tomography image reconstituted in real time is an image with regard to a face orthogonal to a rotational axis (Z axis).

A region in which a CT value falls in a range of equal to or larger than −2051 and less than 0 (mainly air and water) is displayed by black.

A region in which a CT value falls in a range of equal to or higher than 0 and less than 32 (mainly white substantia) is displayed by blue.

A region in which a CT value falls in a range of equal or higher than 32 and less than 40 (mainly gray white substantia) is displayed by blue.

A region in which a CT value falls in a range of equal to or higher than 40 and less than 200 (mainly hemorrhage portion) is displayed by green.

A region in which a CT value falls in a range of equal to or higher than 200 and less than 4048 (mainly bone)is displayed by white.

The region displayed by green is a portion having a high possibility of hemorrhage. An observer can acquire beforehand outline information for cerebral ischemia diagnosis from the tomography image classified into colors in accordance with CT values and displayed in real time.

After scanning is finished, a cerebral ischemia diagnosis assisting processing is started under control of the analysis control portion 241. First, a display direction of the image is corrected by the image display direction correcting portion 242 (S6). As described above, the tomography image reconstituted in real time is an image with regard to a face-orthogonal to the rotating axis (Z-axis). The image display direction correcting portion 242 corrects the display direction of the tomography image reconstituted in real time (hereinafter, referred to as real time tomography image). Actually, the image display direction correcting portion 242 manually or automatically detects the central face separating the left and right cerebral hemispheres from the real time tomography image and generates the tomography image of a face orthogonal to the central face separating the left and right cerebral hemispheres by the multiplanar reconstruction processing (MPR) from the volume data.

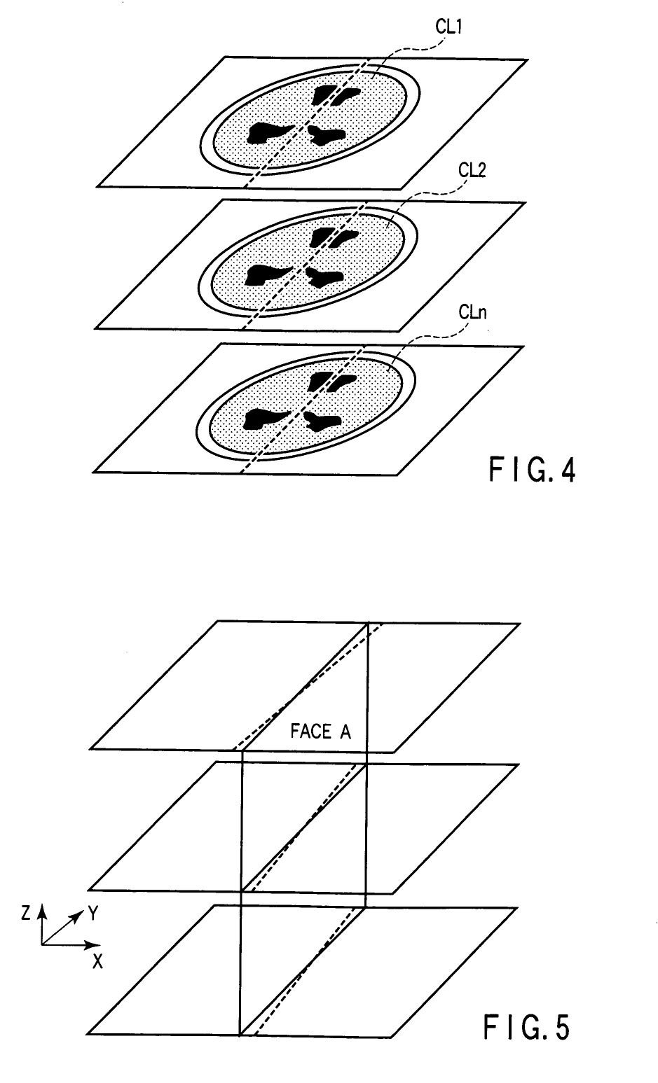

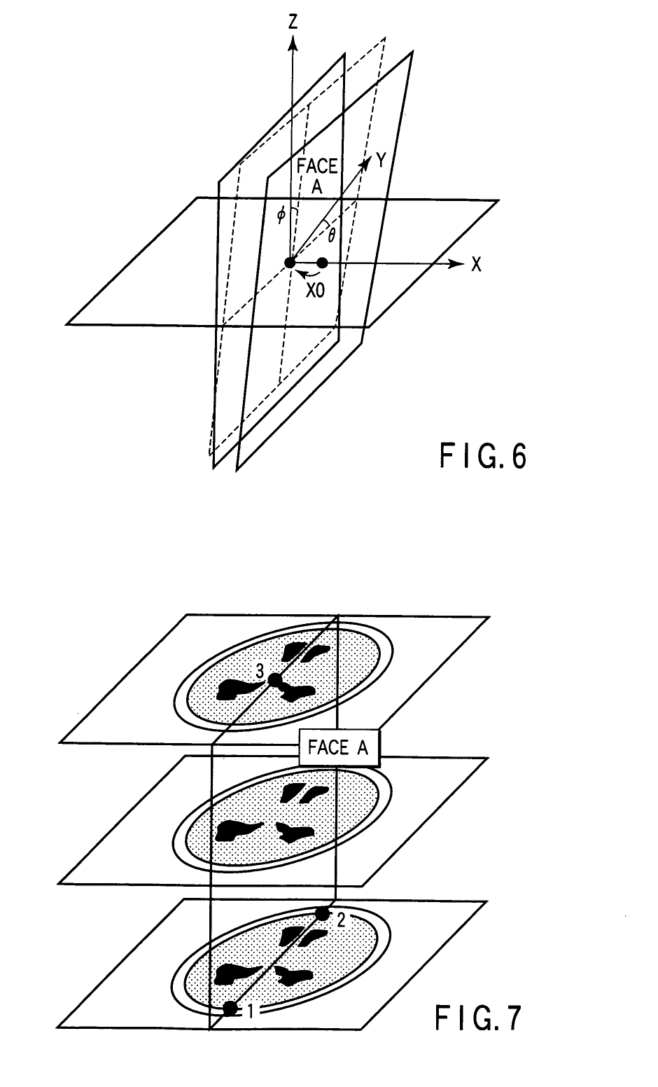

The central face of the left and right cerebral hemispheres can be prescribed by at least two pieces of left and right brain center lines having different positions of Z-axis. The left and right brain center line is a line for separating the left cerebral hemispheres and the right cerebral hemispheres of the brain in the slice face (XY face). A description will be given later of a method of calculating the left and right brain center line. As shown by FIG. 4, n pieces of left and right brain center lines CL1, CL2 . . . CLn are calculated from tomography images of n sheets of slice faces. As shown by FIG. 5, a face A minimizing a total sum of distances (shifts) to the n pieces of left and right brain center lines CL1, CL2 . . . CLn is searched. The face A is prescribed as the left and right brain center face. Here, in order to facilitate an image generating processing thereafter, the volume data is moved and rotated (coordinates transformation) to coincide the left and right brain center face A with a YZ face as shown by FIG. 6. By cutting out an image of XY face from the volume data subjected to coordinates transformation, the tomography image of the slice face in which the left cerebral hemisphere and right cerebral hemisphere are mostly proximate to symmetry can be generated. The brain is partially expanded or contracted by the cerebral ischemia. Therefore, in the diagnosis of the cerebral ischemia, the symmetry in the left and right of the brain is important. When the slice face is inclined to either of the left side and the right side, reliability of the diagnosis based on the symmetry is deteriorated. By generating the tomography image by slicing the face orthogonal to the left and right brain central face, reliability of the diagnosis based on the symmetry is promoted. The left and right hemispheres of the brain are substantially symmetrical with each other and when the face A constituting the center of the symmetry is orthogonal to the slice face (XY face), also the brain illustrated in the image becomes symmetrical in the left and right direction and therefore, by comparing the left and right hemispheres by the observer, a low adsorption region can be discovered or expansion of the brain, narrowing of the ventricle of the brain or the like can be determined. Further, when a line constituting the center of the symmetry in the image resides with Y-axis, the left and right hemispheres are easy to compare. Further, by reforming the respective images such that the face A coincides with the YZ plane, the image can very effectively be read.

Further, although in the above-described, the left and right brain central face is automatically calculated, the face can manually be set. In this case, as shown by FIG. 7, at least two slices of tomography images are generated and the operator designates at least three points on the assumed central faces from respectives of two slices of the tomography images. The left and right brain central face can be calculated from the designated three points. Further, a three-dimensional image of the brain is generated from the volume data and is displayed along with a semitransparent YZ face. By freely moving and rotating the three-dimensional image by manual operation by the operator, the operator may coincide the assumed left and right brain central face to the YZ face.

Next, an explanation will be given of a method of calculating the left and right brain center line. The calculation of the left and right brain center line is an algorism for searching a line at which symmetry between a right half and a left half of an image becomes the highest. Actually, high or low of the symmetry is calculated as cross correlation between the left and right inverted image constituted by folding back the tomography image centering on the assumed left and right brain center line and the tomography image before inversion.

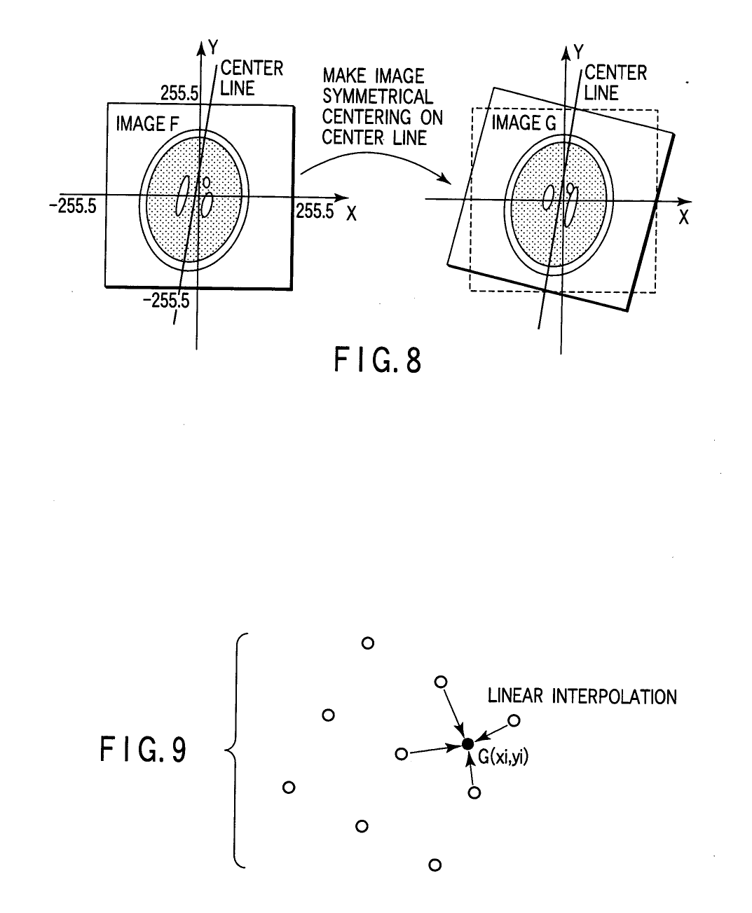

For example, in FIG. 8, assume that an image F is provided with a width of Nx pixels in X direction and a width of Ny pixels in Y direction. In the case of CT image, typically, Nx is 512 the same as Ny.
i={0, 1, . . . , Nx−1}
j={0, 1, . . . , Ny−1}

Here, xi and yj are defined as follows.
xi=[i−(Nx−1)/2]dx
yj=[j−(Ny−1)/2]dy

Incidentally, dx and dy are respectively widths between pixels in x direction and y direction.

Here, an image center line is initially set as a left and right brain center line and an image G is formed by subjecting an image F to left and right inversion centering on brain left and right center line. Further, a point g(xi, yi) on the image in correspondence with a point f(xi, yi) on the image F is calculated. As shown by FIG. 9, g(xi, yi) is calculated from surrounding points by linear interpolation. Further, a correlation coefficient R(f, g) between f(xi, yi) and g(xi, yi) is calculated.
R(f, g)=Sfg/√(Sf*Sg)
Further, Mf=Σf(xi, yi)/(Nx*Ny)
Sf=Σ[{f(xi, yi)−Mf}²]
Mg=Σg(xi, yi)/(Nx*Ny)
Sg=Σ[{g(xi, yi)−Mg}²]
Sfg=Σ[{f(xi, yi)−Mf}*{g(xi, yi)−Mg}]

An equation of the left and right brain center line maximizing the value of the correlation coefficient R(f, g) is calculated by an iteration method.

At the image processing portion 243, a processing similar to that of S5 is carried out for the tomography image (original tomography image) of XY face orthogonal to the left and right brain central face generated by the image display direction correcting portion 242 to determine presence or absence of hemorrhage (S7). That is, the range in which the CT value is equal to or higher than 40 and less than 200 is sampled from the original tomography image and it is determined whether the size of the region is larger than a predetermined size. Specifically, a number of pixels in the region is compared with a threshold. In place of the number of pixels in the range, a rate of the number of pixels having the CT value in the range equal to or higher than 40 and less than 200 as compared with a number pixels having the CT value in the range equal to or higher than 0 and less than 40, or a rate of the number of pixels having the CT value in the range equal to or higher than 40 and less than 200 as compared with a number of pixels having the CT value equal to or higher than 200 may be compared with the threshold respectively in correspondence with therewith.

When the number of pixels in the range is equal to the threshold or larger than the threshold, the region is determined to be a region having a high possibility of hemorrhage and when the number of pixels in the region is smaller than the threshold, the region is determined to have a low possibility of hemorrhage and a high possibility of noise. When the possibility of hemorrhage is determined to be high, a contour line of the line is displayed by being overlapped on the original tomography image by a specific color (S8) and the ischemia diagnosis assisting can be finished.

When there is not present the region in the range having the CT value equal to or higher than 40 and less than 200, or even when the region is present, in the case in which the size of the region is smaller than the predetermined size, the possibility of hemorrhage is determined to be low and the ischemia diagnosis assisting is continued.

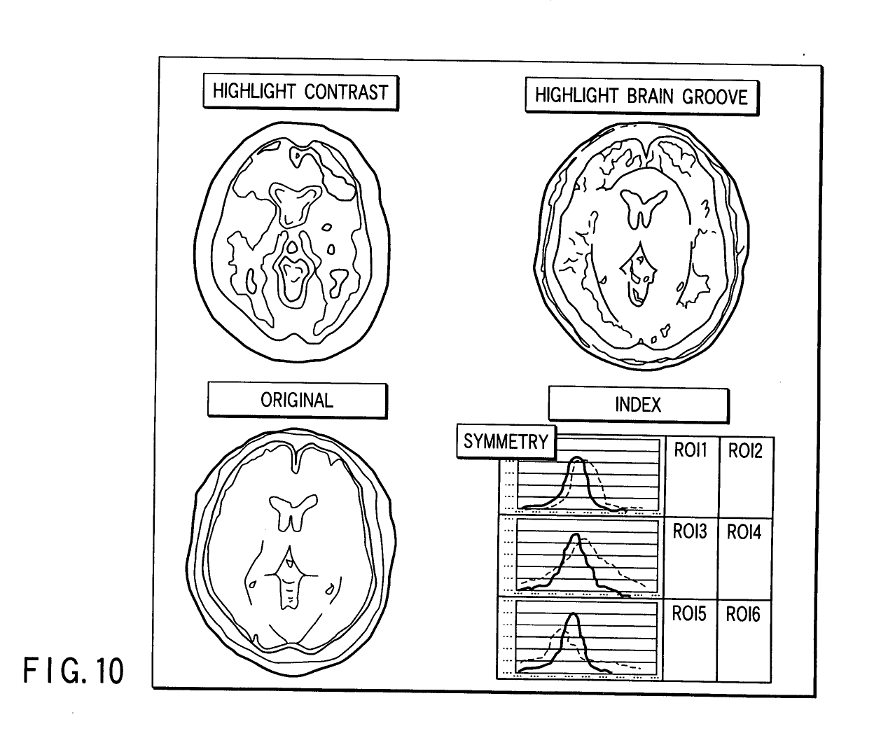

The image processing portion 243 generates a contrast highlighting image and a cerebral sulci highlighting image by processing the original image and the index calculating portion 244 calculates index (index) effective in the brain ischemia diagnosis from the original image (S9, S10) to display as shown by FIG. 10 (S11).

The contrast highlighting image is an image in which the brain tissue region of the original image is classified at least by two kinds of colors in accordance with the CT value. The cerebral sulci highlighting image is an image highlighting the cerebral spinal fluid region of the original image. The index calculating portion 244 calculates the volume ratio between the left cerebral hemisphere region and the right cerebral hemisphere region, the area ratio of the left cerebral hemisphere region and the right cerebral hemisphere region of the original image, and the correlation coefficient between the original image and the left and right inverted image inverted centering on the center line of the original image as useful indices of the brain ischemia diagnosis representing the symmetry between the left cerebral hemisphere and the right cerebral hemisphere.

The contrast highlighting image is an image which is color-coded such that the X-ray low absorption region in the tomography image of CT is easy to observe. For example, as a color scale for facilitating to determine ischemia, the CT value range (20-40) of the brain tissue is classified by three kinds of colors as follows as ranges of objects of coloring.
High (34-40) . . . red
Ordinary (27-33) . . . pink
Low (20-27) . . . blue

Although in the case of a normal head portion image, a distribution of the CT value is substantially symmetrical in the left and right direction, in the ischemia region, in comparison with the opposed side, color in correspondence with the low CT value (blue) is widely distributed. By observing the distribution, the ischemia region can be determined. Further, the CT value is varied by a condition of forming the tomography image. Therefore, the CT value range of the brain tissue and a partition of coloring may automatically be adjusted. As is well known, the CT value is provided with high dependency on a reconstitution function and the tube voltage. Hence, a plurality of combinations of the CT value ranges of the brain tissue and the partitions of coloring are made to correspond to a plurality of combinations of the reconstitution functions and the tube voltages and when the tube voltage and the reconstitution function are read from information accompanied by the image, the CT value range of the brain tissue and the partition of coloring are applied in accordance with the combinations.

There may be adopted a method of automatically determining the CT value range of the brain tissue and the partition of coloring from a CT value histogram. There is formed a histogram representing numbers of pixels for respective CT values of only pixels of the brain tissue while constituting a processing range only by pixels having values within the CT value range of, for example, 5 through 60 which is wide to a degree of permitting a variation in the CT value range of the brain tissue. Further, there is calculated a value of X in which a number of pixels having the certain CT value or larger correspond to 40% of a number of total pixels and the value is made to constitute an upper limit threshold of the region having a high possibility of ischemia (blue region). Although what is detected by the threshold is not strictly the cerebral cortex, the threshold is sufficient for an object of finding the low absorption region (region having low CT value). Here, the abscissa of the histogram designates the CT value and the ordinate designates a rate [%] relative to the number of total pixels and when the left and right histograms are overlapped to display by graphs, a difference between the left and the right ones is facilitated to compare.

The cerebral sulci highlighting image is an image in which the cerebral sulci region is color-coded for observing the cerebral sulci in the tomography image of CT. As the color scale facilitating to determine the cerebral sulci, the coloring object range is constituted by the CT value range (−200 through 20) of the cerebral spinal fluid present at the cerebral sulci and the range is constituted by color scales classified by two kinds of colors as follows.
High (10 through 20) . . . orange
Low (−200 through 9) . . . yellow

In the case of the CT tomography image of the normal head portion, the cerebral sulci is illustrated substantially in left and right symmetry. However, at a vicinity of an ischemia region, the cerebral sulci is narrowed by bringing the brain into tumor and the cerebral sulci is less illustrated than on the opposed side. By observing the illustration, the ischemia region can be determined.

Further, instead of coloring the cerebral sulci, presence of the cerebral sulci may be highlighted by drawing the contour line of the brain. Although in the normal case, by the presence of the cerebral sulci, the contour line becomes a meandering line, at the vicinity of the ischemia region, since the cerebral sulci is not illustrated, the contour line becomes a smooth line without meandering. Here, six lines are formed by dividing the contour line in two divisions in the left and right direction and divided in three of front portion, center portion and rear portion to constitute a total of six divisions and by comparing the length, presence or absence of the cerebral sulci can be represented by a numerical value. Further, a line which is found to be short is colored to highlight a portion at which the cerebral sulci disappears. Thereby, the user can determine the ischemia region.

As indices effective in ischemia diagnosis, the symmetry between the right cerebral hemisphere region and the left cerebral hemisphere region, average CT values of respective six regions (ROI) constituted by dividing a region at a vicinity of a surface of the brain in the circumferential direction centering on the body axis, and distributions of numbers of pixels for respective CT values of six ROI are calculated by the index calculating portion 244. In order to limit the region at the vicinity of the surface of the brain, first, the contour of the brain is sampled. When the contour is defined as a closed curve C and a closed curve constituted by contracting C by about 70% is defined as D, a region in a shape of a circular ring surrounded by C and D is produced. The region is divided into six regions (ROI) by dividing the region in two in the left and right direction and dividing the front, the center and the rear by three to constituted a total of six divisions. Further, average CT values of respectives of six of ROI are calculated. The index calculating portion 244 further calculates a difference between the average CT value of ROI of the front on the right side and the average Ct value of ROI of the front on the left side and when the difference is larger than a predetermined threshold, the index calculating portion 244 constitutes a region having a high possibility of ischemia by the right side front ROI or the left side front ROI having a smaller average CT value and superposes the contour on all or any of the original image, the contrast highlighting image and the cerebral sulci highlighting image. Also with regard to respectives of the central region and the rear region of the region in the circular ring shape, similarly, the average CT values are compared between the left and the right sides and when the difference is larger than the threshold, a region having a high possibility of ischemia is constituted by a region having a small average CT value and the contour is superposed on all or any of the original image, the contrast highlight image and the cerebral sulci highlight image. Further, the respective average CT values of the six regions are numerically displayed.

Here, it is determined statistically by using the pixel number O in the region, a dispersion SD of the CT value and the average CT value M. When a difference is constituted statistically, the contour portion rimming the region is colored to highlight at which region ischemia is present. Thereby, the user can determine the ischemia region.

The symmetry between the right cerebral hemisphere region and the left cerebral hemisphere region is a parameter used in searching the left and right brain center line. The fact that the value of the symmetry exceeds the threshold and is significantly deviated from the normal value suggests that either of the left or right cerebral hemisphere is expanded or contracted. Further, presence of the expansion or the contract is highlighted by displaying a numeral of the symmetry by a color different from that of normal time when the value of the symmetry is equal to or smaller than the threshold.

Further, although in the above-described, the symmetry between the left and the right hemispheres is calculated at the two-dimensional region, the symmetry may be constituted by comparing volumes (three-dimensional regions) of the left and right brain tissues. Left and right three-dimensional regions are switched centering on the left and right central face similar to the case of the two-dimensional region and the correlation coefficient is calculated between the volume before switching and the volume after switching. Further, as symmetry, there may be adopted a difference or a ratio thereof between volumes (or areas) of the left cerebral hemisphere region and the right cerebral hemisphere region. When the difference or the ratio of the volumes or the areas exceeds a predetermined threshold, presence of expansion or contraction is highlighted by displaying the volumes or the areas along with the difference or the ratio by a color which differs from a color when the difference or the ratio is equal to or smaller than the threshold.

Further, the initial slice position of the image displayed on the display screen of FIG. 10 is determined as follows. The control portion 241 detect a slice in which base nuclei having a clinically high value is illustrated by any of followings or any combinations thereof.

A slice 2 cm above a slice at which the upper side of the ear is not seen.

A slice at which the cerebral ventricles are divided in four and each thereof is provided with a specific size or larger.

A slice at which the Silvius fissure is seen by a size to some degree.

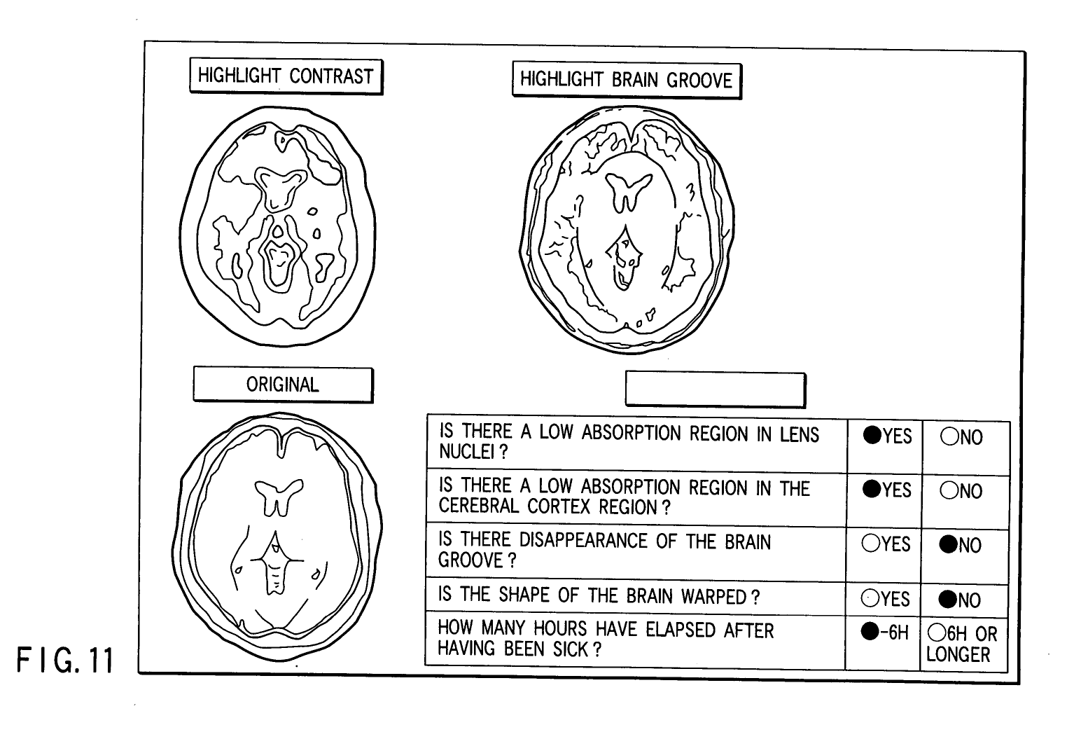

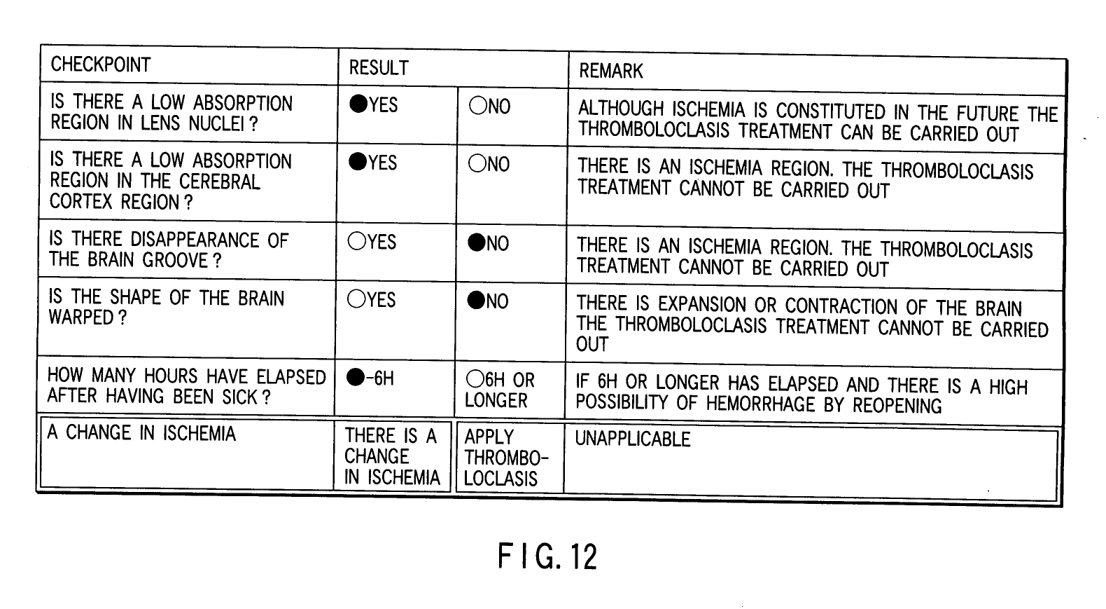

Next, an explanation will be given of diagnosis guide line display by the guideline generating portion 245. A button designated as “guide of diagnosis/treatment” is displayed on, for example, the screen of FIG. 10. By clicking the button, as shown by FIG. 11 and FIG. 12, the guide line of the cerebral ischemia diagnosis. The guide is prepared with a plurality of user check items for the cerebral ischemia diagnosis along with remarks. As items, for example,
Is there a low absorption region in lens nuclei? (remark; although ischemia is constituted in the future, the thromboloclasis treatment can be carried out).
Is there a low absorption region in the cerebral cortex region? (remark; there is an ischemia region. The thromboloclasis treatment cannot be carried out.)
Is there disappearance of the cerebral sulci? (remark; there is an ischemia region. The thromboloclasis treatment cannot be carried out.)
Is the shape of the brain warped? (remark; there is expansion or contraction of the brain. The thromboloclasis treatment cannot be carried out.)
How many hours have elapsed after having been sick? (remark; if 6 H or longer has elapsed and there is a high possibility of hemorrhage by reopening.)

The guide line generating portion 245 is stored with presence or absence of a change in ischemia (ischemia diagnosis result) and applicability (item of caution in treatment) of treatment (for example, thromboloclasis) in correspondence with combinations of answers of the check items. There are displayed presence or absence of a change in ischemia and applicability of treatment in correspondence with combinations of answers of check items.

As described above, according to the embodiment, information useful for the cerebral ischemia diagnosis is provided from the CT image. Therefore, accuracy of the cerebral ischemia diagnosis can be promoted and the effective treatment policy can be determined.

### SECOND EMBODIMENT

An apparatus for aiding the diagnosis and treatment of acute cerebral infarction is described below by referring to the drawings, the apparatus being built according to the present invention. This aiding apparatus for the diagnosis and treatment of acute cerebral infarction treats CT images (spatial distribution of CT values) obtained by an X-ray computed tomography system (CT scanner). In the description given below, it is assumed that the aiding apparatus for the diagnosis and treatment of acute cerebral infarction is incorporated within the X-ray CT scanner. The aiding apparatus for the diagnosis and treatment of acute cerebral infarction can also be constructed separately from the X-ray CT scanner as a standalone apparatus.

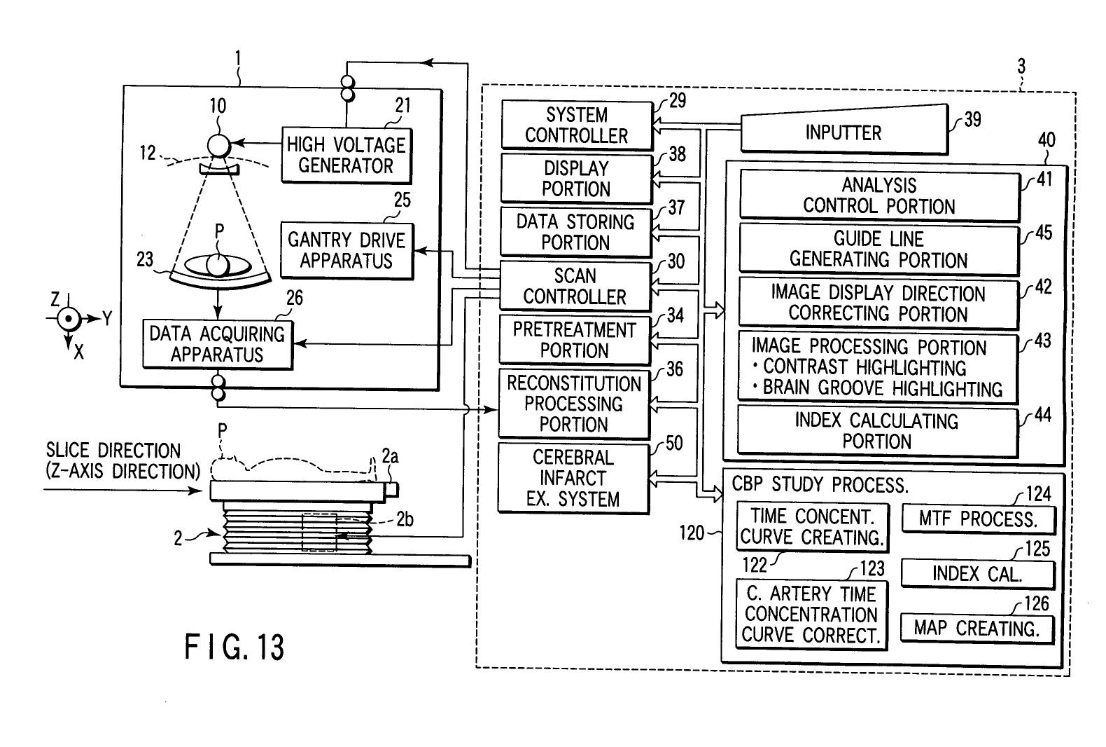

As shown in FIG. 13, the X-ray CT scanner according to the present aspect has a gantry 1 designed to collect projection data about a patent to be examined. The gantry 1 has an X-ray tube 10 and an X-ray detector 23, which are mounted on an annular rotating frame 12 that is rotationally driven by a gantry driver 25. The rotating frame 12 has an opening in its center. The patient P placed on the top plate 2a of a bed 2 is inserted in the opening. The center axis of rotation of the rotating frame 12 is defined to be the Z-axis (in the direction of slices). The plane perpendicular to the Z-axis is defined by two mutually perpendicular axes, or X and Y axes, perpendicular to the Z-axis.

A tube voltage is applied between the anode and cathode of the X-ray tube 10 from a high-voltage generator 21. A filament current is supplied to the filament of the X-ray tube 10 from the high-voltage generator 21. The application of the tube voltage and supply of the filament current produce X-rays. Either a one-dimensional array of detectors or a two-dimensional array of detectors (also known as multi-slice detector system) may be adopted as the X-ray detector 23. Each X-ray detector element has a square sensitive surface, for example, measuring 0.5 mm×0.5 mm. For example, 916 X-ray detector elements are arrayed in the direction of channel. Where 40 such arrays, for example, are juxtaposed in the slice direction, a two-dimensional array of detectors is obtained. Where there is only one array, a one-dimensional array of detectors is obtained.

A data collection unit 26, generally known as a data acquisition system (DAS), converts a signal produced from the detector 23 for each channel into a voltage signal, amplifies it, and further converts it into a digital signal. This data (raw data) is supplied to a computer unit 3 that is outside the gantry. The computer unit 3 has a preprocessing portion 34 that performs corrective processing such as sensitivity correction on the raw data output from the data acquisition unit 26 and outputs projection data. This projection data is sent to the data storage unit 37 of the computer system 3 and stored there.

The computer system 3 has the aforementioned preprocessing portion 34 and storage unit 37. In addition, the system has a system controller 29, a scan controller 30, a reconstruction processing portion 36, a display portion 38, an input device 39, an apparatus 40 (cerebral ischemia analysis (CT-DE) apparatus) for aiding the diagnosis of cerebral ischemia, a cerebral infarction expert system 50, and a CBP study processing portion 120. The input device 39 has a mouse, a keyboard, and so on.

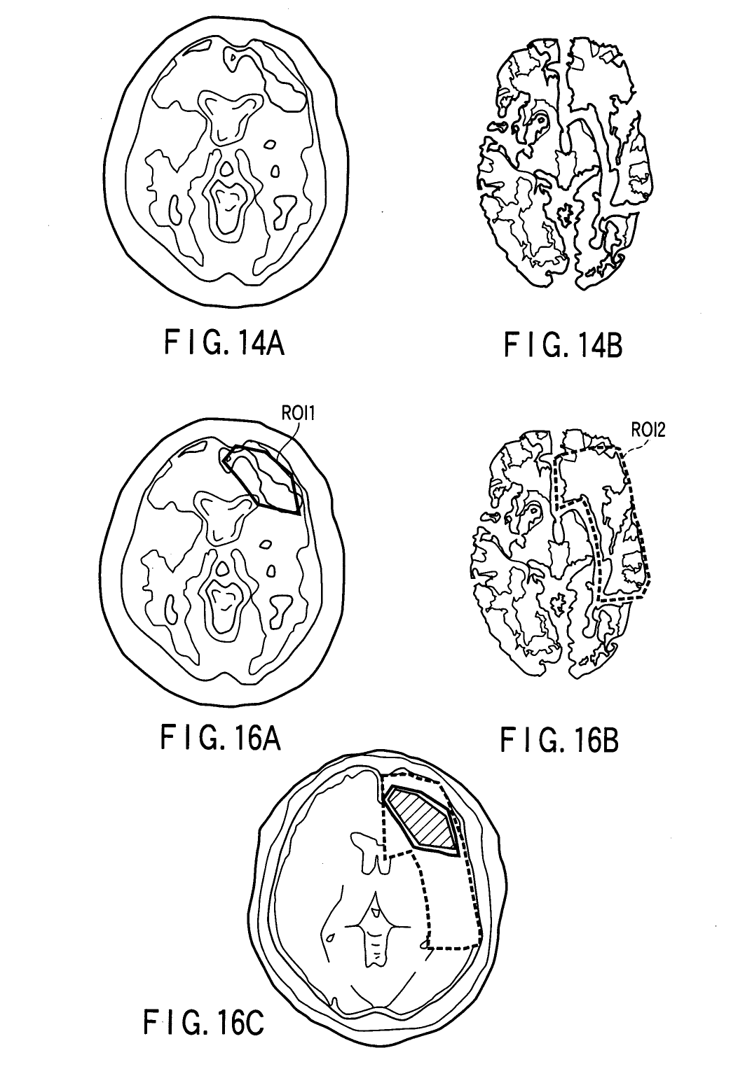

The apparatus 40 for aiding the diagnosis of cerebral ischemia processes the spatial distribution (CT images) of CT values reconstructed by the reconstruction processing portion 36, for example, and creates contrast-enhanced images and cerebral sulci-enhanced images as information useful for aiding the diagnosis of cerebral ischemia. This will be described in further detail later. As shown in FIG. 14A, a contrast-enhanced image is produced by classifying the cerebral tissue regions on the original CT images into at least two categories according to CT value and giving different colors to these different categories. A cerebral sulci-enhanced image is produced by enhancing the cerebrospinal liquid regions on the original images. An ischemic region appears as a low CT value region. A low CT value region can be extracted as a doubtful ischemic region from the contrast-enhanced and cerebral sulci-enhanced images.

The CBP study processing portion 120, which will be described in detail later, images the manner how the contrast medium passes by dynamic CT technology to thereby produce successive images, calculates CBP, CBV, MTT, and Err for each pixel as indexes quantitatively representing the dynamic state of the blood flow through blood capillaries within the cerebral tissues, and creates a spatial map (cerebral blood flow images) of the indexes. An example of the CBP map is shown in FIG. 14B. CBP indicates the flow rate of blood [ml/100 ml/min] within the blood capillaries within the cerebral tissues per unit volume and per unit time. CBV indicates the amount of blood [ml/100 ml] per unit volume of the cerebral tissues. MTT indicates the mean transit time [seconds] in which blood passes through a blood capillary. Err indicates the total sum of residual errors or the square root of the sum of the squares of residual errors when a transfer function is approximated. A low blood flow region can be extracted as a doubtful ischemic cerebrovascular disease from the map (cerebral blood flow images) of the indexes quantitatively representing the dynamic state of the blood flow through the blood capillaries within the cerebral tissues.

In the present aspect, two different kinds of images are provided in this way. That is, contrast-enhanced images of low CT value regions are obtained by subjecting the spatial distribution of CT values (CT images) to threshold-value processing or clustering processing, the distribution being taken by non-contrast enhanced CT scanning. A map (brain blood flow images) of CBP and so on that quantitatively represents the dynamic state of the blood flow through blood capillaries within the cerebral tissues is offered, the blood flow being obtained by processing successive images taken by dynamic CT scanning. The maximum feature of the present aspect is that a frame line indicating the contour of a low CT value region (ischemic region) (first ROI) identified from contrast-enhanced images and a frame line indicating the contour (second ROI) of a low blood flow region identified from the cerebral blood flow images are superimposed on the CT images and displayed side by side. That is, the ischemic region as viewed from simple CT values of the unenhanced CT image is not coincident with the ischemic region as viewed from the quantitative values indicating the dynamic state of the blood flow through the blood capillaries within the cerebral tissues. Normally, the latter region that is hypersensitive to ischemic cerebrovascular diseases is wider than the former ischemic region as viewed from simple CT values. The former ischemic region that can be judged from simple CT values is often irrecoverable. Furthermore, treatment of ischemia involves a danger of intracerebral hemorrhage. In order to decide to perform a treatment of ischemia in spite of the presence of such a danger, it is necessary that some degree of recoverableness (therapy effects) can be expected. The differential region between the low CT value and low blood flow regions can be regarded as a region that can be recovered by a treatment. This is essential useful information in making a decision as to whether a treatment is performed.

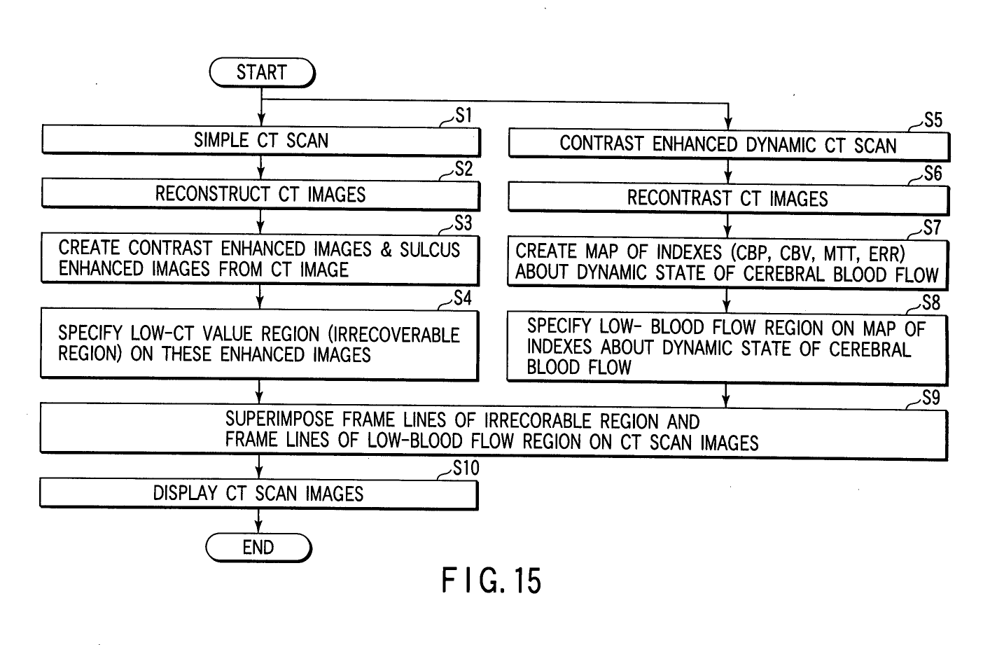

The procedure of sequence of operations according to the present aspect is illustrated in FIG. 15. This sequence of operations operates under control of the cerebral infarction expert system 50. In the present aspect, the CT scanning for collecting data includes two modes, i.e., simple CT scanning S1 and contrast dynamic CT scanning S5. Typically, a simple CT scan is performed on certain axial slices regarding the head of the patient. At arbitrary time after the end of the simple CT scan, a contrast medium is injected into the patient, and a dynamic CT scan on the same certain slices is started. The slices is set in a region including the bansal ganglia.

A simple CT scan means a scanning operation to collect one or more sets of projection data about 360° or 180°+fan angle of certain slices without using a contrast medium. On the other hand, in a dynamic CT scan, a contrast medium is injected to investigate how the medium varies with time. At an appropriate time after the injection, acquisition of sets of projection data about 360° or 180°+fan angle about certain slices is started. This operation is repeated consecutively during a period that ends when the contrast medium almost completely flows out of the certain slices, for example.

The spatial distribution (CT images) of the CT values of the certain slices is reconstructed by the reconstruction processing portion 36 based on the projection data collected in the simple CT scan S1 (step S2). The apparatus 40 for aiding the diagnosis of cerebral ischemia processes the CT images and creates contrast-enhanced images and cerebral sulci-enhanced images (step S3). In each contrast-enhanced image, the cerebral tissue regions of the original images are classified into at least two categories according to CT value, and different colors are given to these categories. This emphasizes the low CT region corresponding to an ischemic region. The cerebral sulci-enhanced image is the image enhancing the cerebrovascular liquid regions of the original image.

This emphasizes the cerebral sulci region that has disappeared due to ischemia.

The contrast-enhanced images are selectively displayed on the display portion 38, together with the cerebral sulci-enhanced images. A frame line indicating the contour (first ROI) of a low CT value region (irrecoverable region) is specified on the contrast-enhanced or cerebral sulci-enhanced image when the input device 39 is operated by a human operator (step S4). An example of the first ROI is shown in FIG. 16A. The method of specifying the first ROI is not limited to manual inputting. Rather, the ROI may be specified from the CT images by threshold value processing or clustering processing. Data that defines the first ROI is stored in the data storage portion 37, along with the contrast-enhanced, cerebral sulci-enhanced, and original CT images.

Based on the projection data collected by the contrast dynamic CT scan (S5), a sequence of CT images about the same certain slices as used when the simple CT scan was performed but taken at different times is reconstructed by the reconstruction processing portion 36 (step S6). The CBP study processing portion 120 processes the CT images, finds the indexes CBP, CBV, MTT, and Err quantitatively indicating the dynamic state of the local blood flow within cerebral tissues, i.e., the dynamic state of blood flow through the blood capillaries within the local tissue, for each pixel, and creates maps of these indexes (cerebral blood flow image) (S7). The map of CBP and the map of CBV, MTT, or Err are selectively displayed on the display portion 38. A frame line indicating the contour of a low blood flow region (second ROI) is specified on the map of CBP when the input device 39 is operated by the operator (S8). An example of the second ROI is displayed in FIG. 16B. The method of specifying the second ROI is not limited to manual inputting. The ROI may be specified from any map by threshold value processing. Data that defines the second ROI is stored in the data storage portion 37, together with CBP maps and so on.

Then, in step S9, the cerebral infarction expert system 50 superimposes the low CT value region (first ROI) and low blood flow region (second ROI) onto the CT images reconstructed in step S2, as shown in FIG. 16C (S9) and displayed side by side on the display portion 38 (S10). The cerebral infarction expert system 50 may display the number of pixels (area) within the first ROI, the number of pixels (area) within the second ROI, and the number of pixels (area) within a differential region together with the images. The differential region is the difference between the second and first ROIs. Furthermore, the ratio of the number of pixels (area) within the first ROI to the number of pixels (area) within the second ROI may be found and displayed.

The diagnosis of acute cerebral infarction and treatment policy can be determined effectively and quickly by using two kinds of images (i.e., images of CT values and cerebral blood flow images) and comparing the low CT value and low blood flow regions in this way.

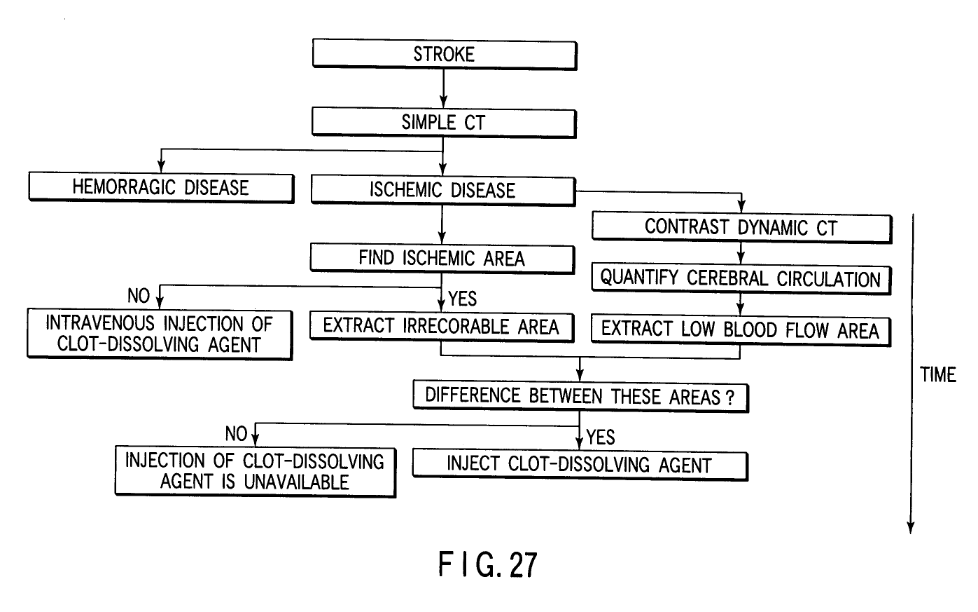

Next, diagnosis of cerebral ischemia and diagnosis of cerebral blood flow are separately described in detail. A typical procedure of operations for diagnosis of acute cerebral infarction and determination of treatment policy in association with the present aspect is shown in FIG. 27 as a reference example.

As shown in FIG. 13, the apparatus 40 for aiding the diagnosis of cerebral ischemia has a cerebral ischemia analysis and control portion 41, an image display direction correcting portion 42, an image processing portion 43, an index calculating portion 44, and a guidance generating portion 45. The image display direction correcting portion 42 corrects the direction of display of the CT images created incipiently from volume data by the image processing portion 43. Practically, the image display direction correcting portion 42 manually or automatically detects the center plane that separates the right and left cerebral hemispheres from the CT images created incipiently, and creates CT images of planes vertical to the center plane separating the right and left cerebral hemispheres from volume data by cross section converting processing (MPR).

The image processing portion 43 processes the CT images (also referred to as the original images) created by the image display direction correcting portion 42 to make a decision as to whether there is a hemorrhage or not. In addition, the image processing portion 43 creates contrast-enhanced and cerebral sulci-enhanced images. The original images are displayed on the display portion 38 together with the contrast-enhanced and cerebral sulci-enhanced images under control of the system controller 29 or CT-DE control portion 41. In each contrast-enhanced image, the cerebral tissue regions of the original image are classified into at least two categories according to CT value, and different colors are given to these categories. The cerebral sulci-enhanced image is the image enhancing the cerebrospinal liquid areas of the original image. The index calculating portion 44 calculates indexes useful for the diagnosis of cerebral infarction, the indexes indicating the symmetry between the right and left cerebral hemispheres. Examples of these indexes are the volume ratio between the right and left cerebral hemispheres, the area ratio between the right and left cerebral hemispheres on the original images, and the correlation coefficients between the original image and each of the right and left inverted images on the opposite sides of the center line of the original image. The guidance generating portion 45 makes a decision as to whether the treatment (e.g., thrombolytic therapy (clot-dissolving therapy) is advisable or not, based on the combination of diagnostic results owing to user's judgment.

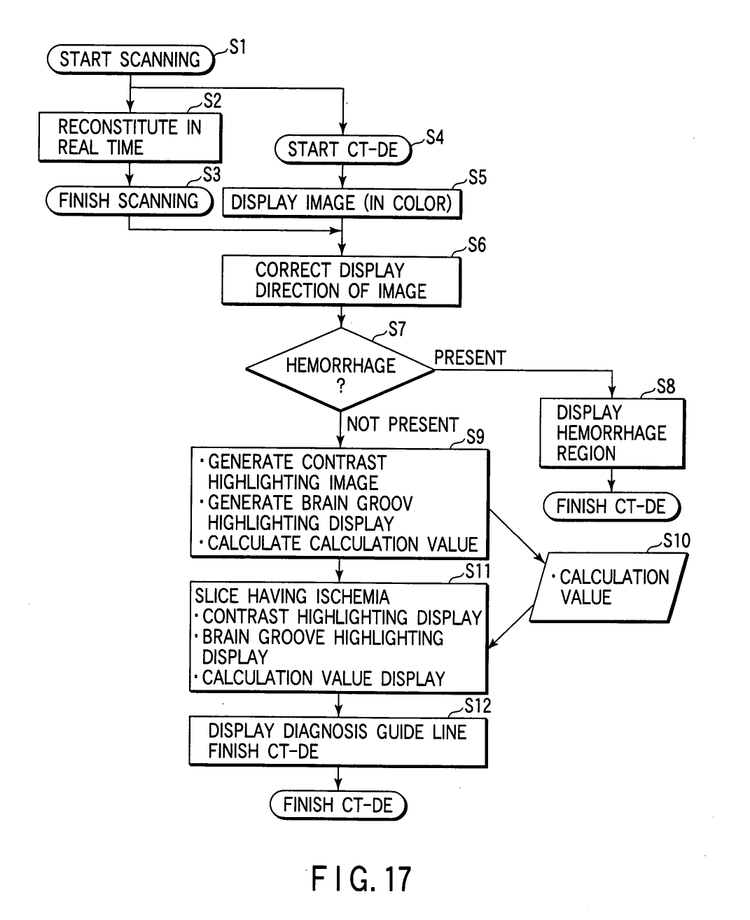

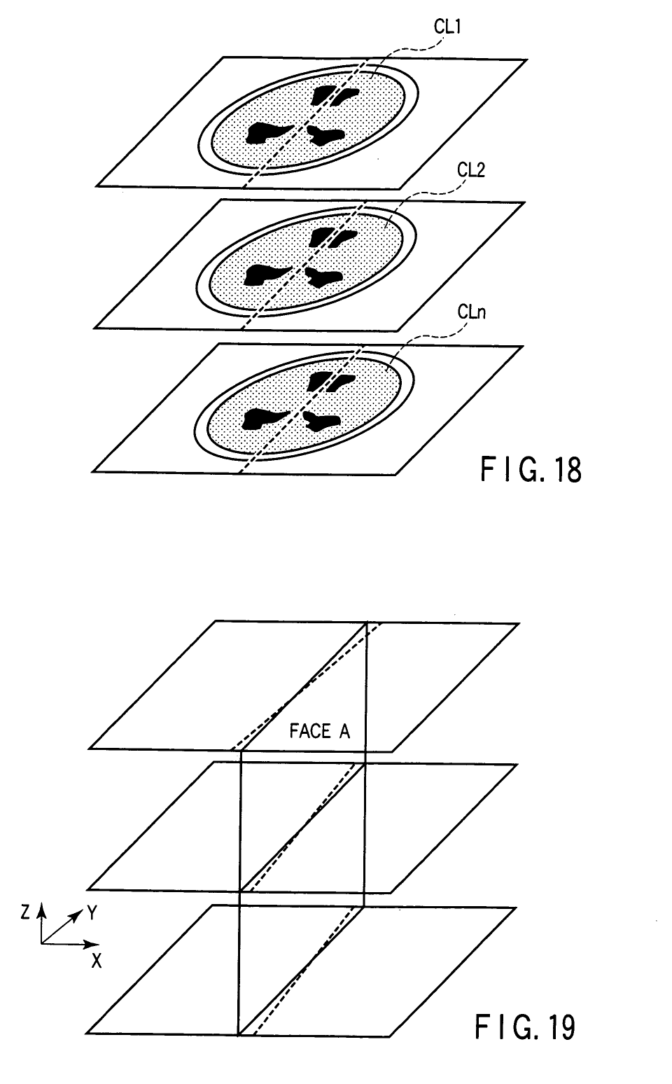

FIG. 17 illustrates the flow of the processing for aiding the diagnosis of cerebral ischemia according to the present aspect. First, a scan is started (S1). This scan is a helical scan or volume scan of the head of the patient. Whenever projection data corresponding to an angle necessary to reconstruct one tomographic image is collected, tomographic images are reconstructed in turn concurrently with the scan (S2). This technique of reconstructing tomographic images in turn concurrently with the scan is a well-known technique and known as real-time CT or CT fluoroscopy. When collection of data from a predetermined range including the head of the patient is completed, the scan ends (S3).

Simultaneously with the start of the scan, the functions for cerebral infarction analysis (CT-DE) are activated by the analysis and control portion 41 (S4). The image processing portion 43 under control of the analysis and control portion 41 displays the CT images reconstructed by real-time CT in five colors, for example, to facilitate finding a bleeding region (S5).

The CT image reconstructed on a real-time basis is an image regarding a plane that is vertical to the axis of rotation (Z-axis).

Regions (principally, air and water) having CT values ranging from −2051 to 0 are displayed in black. Regions (mainly, white matter) having CT values ranging from 0 to 32 are displayed in blue. Regions (chiefly, gray matter) having CT values ranging from 32 to 40 are displayed in blue. Regions (mainly, bleeding portion) having CT values ranging from 40 to 200 are displayed in green. Regions (chiefly, bones) having CT values ranging from 200 to 4048 are displayed in white.

Regions displayed in green are portions which are likely to bleed. The observer can previously gain rough information for diagnosis of cerebral ischemia from the CT images displayed in real time in different colors according to CT value.

After the end of the scan, processing for aiding the diagnosis of cerebral ischemia is started under the control of the analysis and control portion 41. First, the direction of display of image is corrected by the image display direction correcting portion 42 (step S6). As mentioned previously, the CT images reconstructed in real time are images about planes vertical to the axis of rotation (Z-axis). The image display direction correcting portion 42 corrects the direction of display of CT images (hereinafter referred to as real-time CT images) reconstructed in real time. Practically, the image display direction correcting portion 42 manually or automatically detects the center plane that separates the right and left cerebral hemispheres from the real-time CT images, creates CT images about planes vertical to the center plane separating the right and left cerebral hemispheres from volume data by cross section conversion processing (MPR).

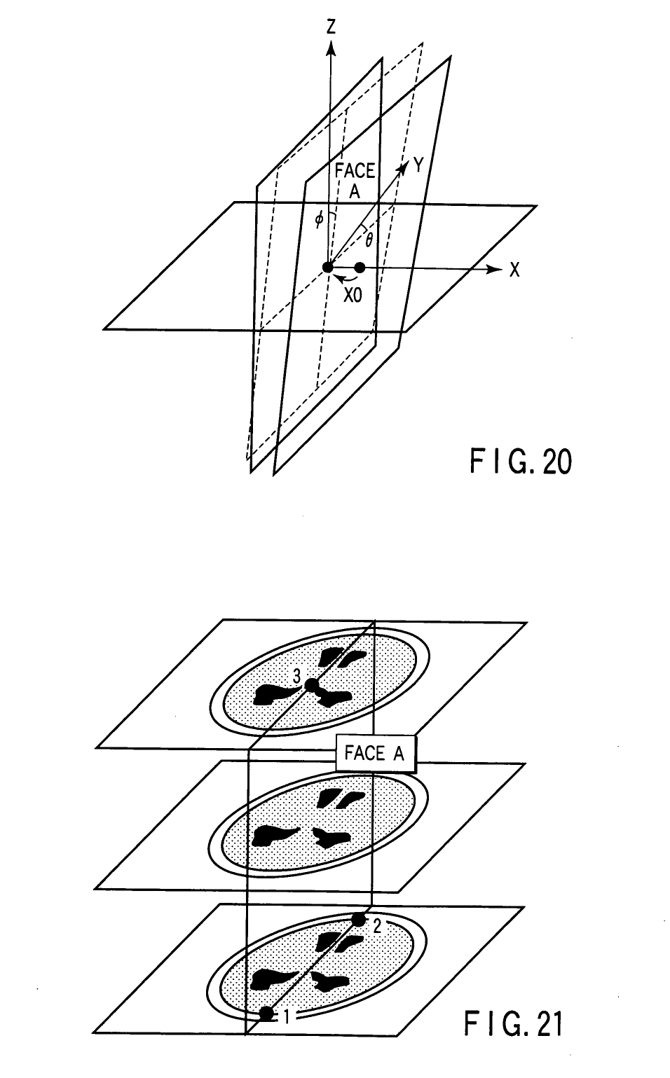

The center plane between the right and left cerebral hemispheres can be defined by at least two center lines between the right and left cerebral hemispheres, the center lines being different in Z-axis position. Each center line between the hemispheres is a line separating the right and left cerebral hemispheres within a slice plane (XY-plane). A method of calculating the center lines between the right and left cerebral hemispheres is described later. As shown in FIG. 18, n center lines CL1, CL2, . . . , CLn between the right and right cerebral hemispheres are calculated from CT images of n slice planes. As shown in FIG. 19, a plane A at which the total sum of distances (deviations) to these n center lines CL1, CL2, . . . , CLn between the right and right cerebral hemispheres is smallest is searched for. This plane A is defined as the center plane between the right and left cerebral hemispheres. In this aspect, to facilitate subsequent processing for creating images, the volume data is moved and rotated (coordinate transformation) as shown in FIG. 20 to bring the center plane A between the hemispheres into agreement with the YZ-plane. A CT image of a slice plane at which the right and left cerebral hemispheres show a maximum degree of symmetry can be created by extracting images on the XY-plane from the coordinate-transformed volume data. Due to cerebral ischemia, the brain partially swells or shrinks. Therefore, in diagnosis of cerebral ischemia, the symmetry between the right and left cerebral hemispheres is important. When the slice plane is tilted right or left, the reliability of the diagnosis based on the symmetry deteriorates. This reliability of the diagnosis based on the symmetry is improved by creating CT images using planes vertical to the center plane between the right and left cerebral hemispheres as slices. The right and left cerebral hemispheres are almost symmetric. If the plane A forming the center of symmetry is vertical to the slice plane (XY-plane), the brain depicted within the image will be symmetric right and left. Therefore, the diagnostic reader can find a low absorption region or judge swelling of the brain or narrowing of cerebral ventricles by comparing the right and left cerebral hemispheres. Furthermore, if a line forming the center of symmetry within the image is coincident with the Y-axis, it is easy to compare the hemispheres. Consequently, each image is re-created such that the plane A agrees with the YZ-plane. This is very effective for diagnostic reading.

In the description provided above, the center plane between the right and left cerebral hemispheres is automatically calculated. It may also be set manually. In this case, as shown in FIG. 21, CT images of at least two slices are created. At least three points assumed by the operator and lying on the center plane are specified from the CT images of the two slices. The center plane between the cerebral hemispheres can be computed from the specified three points. Furthermore, a three-dimensional image of the brain is created from volume data and displayed together with the semitransparent YZ-plane. The center plane between the cerebral hemispheres assumed by the operator may be brought into coincidence with the YZ-plane by moving and rotating the three-dimensional image arbitrarily. This is achieved by operator's manual operations.

A method of calculating the center line between the right and left cerebral hemispheres is next described. The calculation of the center line between the hemispheres is an algorithm for searching for a line on which the degree of symmetry between the right and left halves of the image is highest.

Practically, the degree of symmetry is found as the cross-correlation between the horizontally inverted image obtained by folding back each CT image about the assumed center line between the cerebral hemispheres and each CT image yet to be inverted.

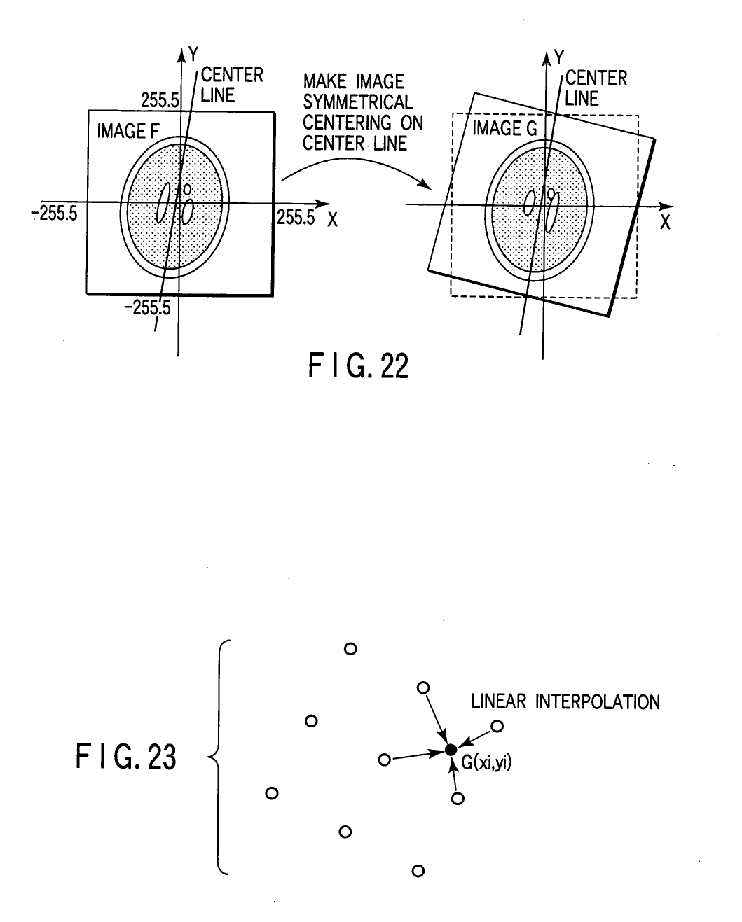

For example, as shown in FIG. 22, it is assumed that an image F has a width given by Nx pixels in the X-direction and a width given by Ny pixels in the Y-direction. In the case of a CT image, Nx is typically the same as Ny, i.e., 512.
i=[0, 1, . . . , Nx−1]
j=[0, 1, . . . , Ny−1]
xᵢ and yⱼ are now defined as follows.
   xᵢ=[i−(Nx−1)/2]dx
   yᵢ=[j−(Ny−1)/2]dy
   where dx and dy are the interpixel widths in the X- and Y-directions, respectively.

The center line of the image is incipiently set as the center line between the right and left cerebral hemispheres. The image F is inverted horizontally about the center line between the hemispheres, thus creating an image G. Then, a point g(xᵢ, yᵢ) which is on the image G and corresponds to a point f(xᵢ, yᵢ) lying on the image F is found. g(xᵢ, yᵢ) is found from surrounding points by linear interpolation as shown in FIG. 21. The correlation coefficient R(f, g) between f(xᵢ, yᵢ) and g(xᵢ, yᵢ) is then found.

$$R ( f , g ) = \frac{Sfg}{\sqrt{Sf * Sg}}$$

where

$$\begin{matrix}Mf = \frac{\sum f ( {x}_{i} , {y}_{i} )}{Nx * Ny} \\ Sf = \sum [ {\{ f ( {x}_{i} , {y}_{i} ) - Mf \}}^{2} ] \\ Mg = \frac{\sum g ( {x}_{i} , {y}_{i} )}{Nx * Ny} \\ Sg = \sum [ {\{ g ( {x}_{i} , {y}_{i} ) - Mg \}}^{2} ] \\ Sfg = \sum [ \{ f ( {x}_{i} , {y}_{i} ) - Mf \} * \{ g ( {x}_{i} , {y}_{i} ) - Mg \} ]\end{matrix}$$

A formula that expresses the center line between the right and left cerebral hemispheres and maximizes the value of the correlation coefficient R(f, g) is found by an iterative method.

The image processing portion 43 processes the CT images (original CT images) on the XY-plane vertical to the center plane between the cerebral hemispheres in the same way as in step S5 to judge whether there is a bleeding (S7), the CT images being created by the image display direction correcting portion 42. In particular, the processing portion extracts an area having a range of CT values indicating bleeding (e.g., from 40 to 200) from the original CT images and makes a decision as to whether the size of this area is greater than a given size. More specifically, the number of pixels within this area is compared with threshold values. Instead of the number of pixels in this area, the ratio of the number of pixels having CT values lying in a CT value range (e.g., 40 to 200) indicating its bleeding to the number of pixels having CT values lying in a CT value range indicating a cerebral tissue (e.g., from 0 to 40) may be compared with a corresponding threshold value. Alternatively, the ratio of the number of pixels having CT values lying in the CT value range (e.g., from 40 to 200) indicating the bleeding to the number of pixels having CT values lying in a CT value range (e.g., more than 200) indicating bones may be compared with a corresponding threshold value.

When the number of pixels in this area is equal to or greater than the threshold value, this area is judged to be a doubtful bleeding area. When the number of pixels in this area is less than the threshold value, this area is judged to be a less doubtful bleeding area and be produced possibly by noise. Where the result of the decision is that there is a high possibility of bleeding, the contour line of this area is displayed in a certain color and superimposed on the original CT images (S8). Thus, the aid to the diagnosis of ischemia can be ended.

When any area having CT values lying in the CT value range (from 40 to 200) indicating bleeding does not exist, or when it exists but the size of the area is smaller than a given size, the result of the decision is that the possibility of bleeding is low. The aid to the diagnosis of ischemia is continued.

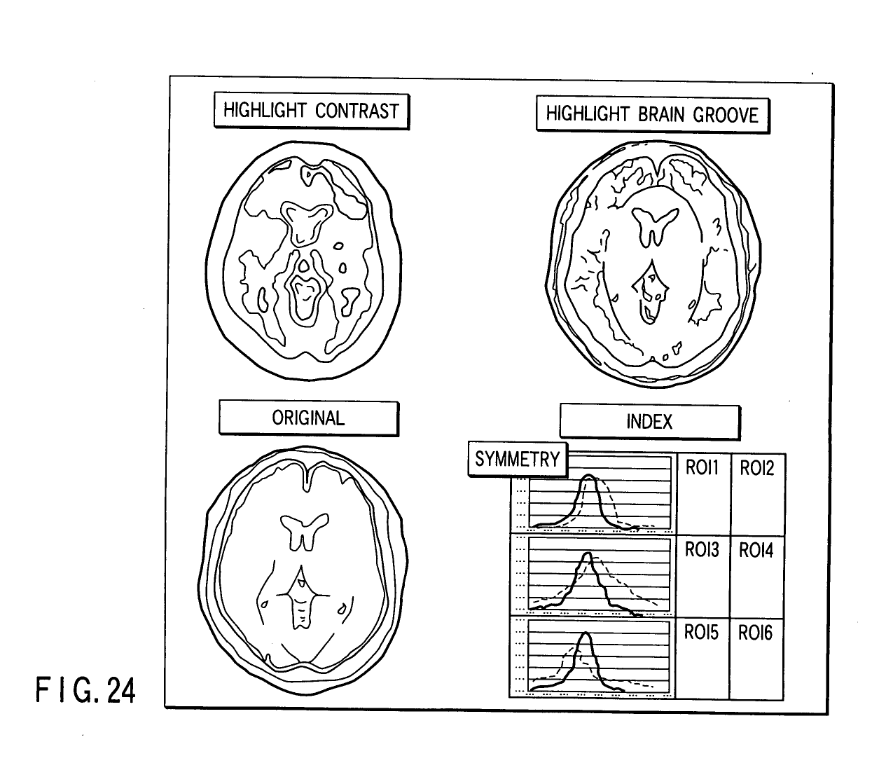

The image processing portion 43 processes the original images and creates contrast-enhanced and cerebral sulci-enhanced images. The index calculating portion 44 calculates indexes effective for the diagnosis of cerebral ischemia from the original images (S9 and S10) and displays them as shown in FIG. 24 (S11).

The contrast-enhanced image is an image in which cerebral tissue areas of the original image are classified into at least two categories according to CT value and displayed in different colors. The cerebral sulci-enhanced image is an image in which cerebrospinal liquid areas of the original image are classified into at least two categories according to CT value and displayed in different colors. The index calculating portion 44 calculates the volume ratio between the right and left cerebral hemispheres, the area ratio between the right and left cerebral hemispheres on the original image, and the correlation coefficients of the original image to the right and left inverted images on the opposite sides of the center line of the original image, for example, as indexes which are useful for the diagnosis of cerebral ischemia and indicate the symmetry between the cerebral hemispheres.

The contrast-enhanced image is a color-coded image to facilitate observing X-ray low absorption areas on the CT image. For example, as a color scale facilitating the judgment on ischemia, the total range of the CT values of cerebral tissues (from 20 to 40) is classified into three categories and different colors are given to them as follows.
higher (34 to 40): red
moderate (27 to 33): pink
lower (20 to 26): blue

In the case of an image of a normal head, the distribution of CT values is almost symmetric right and left. In an ischemic region, the color (blue) indicating low CT values is distributed over a wider area compared with the opposite side. The ischemic region can be judged by observing this. CT values vary according to the conditions under which tomographic images are created. Accordingly, the classification of the CT values of cerebral tissues and color coding may be automatically adjusted by the following method. As is well known in the art, CT values depend heavily on the reconstruction function and on the tube voltage. Accordingly, plural combinations of CT value ranges of cerebral tissues and color-coding schemes are made to correspond to plural combinations of reconstruction functions and tube voltages. When tube voltage and reconstruction function are read in from ancillary information about an image, the CT value range of cerebral tissues and color-coding scheme are applied according to the combination.

A method of automatically determining the ranges of CT values of cerebral tissues and color-coding scheme from a CT value histogram may be adopted. In an image, only pixels having CT values lying within a wide range that tolerates variations in CT values of cerebral tissues such as from 5 to 60 are assumed to lie in a range to be processed. As this range, a histogram indicating the number of pixels at each CT value of only pixels in the cerebral tissues is created. The number of pixels having CT values exceeding some CT value X is found. The value of X which establishes the relation that this number of pixels is 40% of the total number of pixels of the cerebral tissues is then found. This X is taken as the upper threshold value of the region (blue region) that is likely to be ischemic. Strictly speaking, what is detected using this threshold value is not cortex, but this threshold value suffices to find a low absorption area having low CT values. In the histogram, CT value is plotted on the horizontal axis and the ratio (%) of the first-mentioned number of pixels to the total number of pixels is on the vertical axis. Where right and left histograms are superimposed and graphically represented, it is easy to compare the right and left portions. That is, it is easy to see the difference between them.

A cerebral sulci-enhanced image is an image in which cerebral sulci regions are color-coded to permit observation of the cerebral sulci on the CT image. To obtain a color scale that facilitates judging cerebral sulci, the total range (−200 to 20) of CT values of cerebrospinal liquid existing in the cerebral sulci is classified into two categories and different colors are given to these categories as given below to obtain a color scale, for example.
higher (10 to 20): orange
lower (−200 to 9): yellow

In the case of a CT image of a normal head, cerebral sulci are depicted almost symmetrically right and left. However, near an ischemic region, the brain swells, narrowing the cerebral sulci. As a result, the cerebral sulci on this side are less depicted than the other side. By observing this, the ischemic region can be judged.

Instead of color-coding the cerebral sulci and making a contrast enhancement, the contour line of the brain may be drawn to emphasize the existence of the cerebral sulci. In a normal case, the contour line makes a serpentine line because of the existence of cerebral sulci. Near an ischemic region, the cerebral sulci are not depicted and so the contour line is not a serpentine line but a smooth line. As an example, the contour line is bisected right and left. Also, the line is trisected into front, middle, and rear portions. In total, 6 line segments are created. By comparing their lengths, the presence or absence of cerebral sulci can be expressed by numerical values. Then, a color is given to shorter line segments to emphasize the portions where the cerebral sulci have disappeared. This permits the user to judge the ischemic region.

The index calculating portion 44 calculates indexes effective for diagnosis of ischemia, i.e., the degree of symmetry between the right and left cerebral hemispheres, the average CT value of 6 ROIs produced by dividing a region close to the cerebral surface circumferentially about the body axis, and the distribution of the number of pixels in each of the 6 ROIs for each individual CT value. To restrict this region close to the cerebral surface, the contour of the brain is first extracted. This contour is taken as a closed curve C. A closed curve obtained by reducing the curve C to about 70% is indicated by D. An annular region surrounded by C and D is formed. This region is bisected right and left and trisected into front, middle, and rear portions. In total, 6 regions (ROIs) are produced. The average CT value of each of these 6 ROIs is calculated. Furthermore, the index calculating portion 44 calculates the difference between the average CT value of the right front ROI and the average CT value of the left front ROI. When the difference is greater than a given threshold value, the right front ROI or the left front ROI having a smaller average CT value is regarded as a region that is likely to be ischemic. Its contour is superimposed on all or any of the original image, contrast-enhanced image, and cerebral sulci-enhanced image. Similarly, with respect to each of central and rear areas of the annular region, the left and right portions are compared in terms of average CT value. When the difference is in excess of the threshold value, the area having a smaller average CT value is regarded as an area that is likely to be ischemic. Its contour is superimposed on all or any of the original image, contrast-enhanced image, and cerebral sulci-enhanced image. In addition, the numerical value of the average CT value of each of the 6 ROIs is displayed.

A statistical judgment is made using the dispersions SDs of the numbers of pixels O in the regions and CT values and their average CT values M. Where there is a statistical difference, the contour portion fringing the region is colored, emphasizing what regions are ischemic. This permits the user to judge the ischemic regions.

The degree of symmetry between the right and left cerebral hemispheres is a parameter used in searching for the center line between the cerebral hemispheres. Where the value of the degree of symmetry exceeds the threshold value and deviates greatly from normal values, it is suggested that either right or left cerebral hemisphere has swollen or shrunk. In this case, the numerical value of the degree of symmetry is displayed in a color different from the colors used for normal state, or less than the threshold value.

In this way, the presence of swelling or shrinkage is emphasized.

In the description above, the degree of symmetry between the right and left cerebral hemispheres is calculated in a two-dimensional region of a CT image. A comparison may be made in a volume (three-dimensional region) of the right and left cerebral tissues. In the same way as in the two-dimensional scheme, right and left 3D regions on the opposite sides of the center plane between the cerebral hemispheres are interchanged. The correction coefficient with the volume not yet interchanged is calculated. In addition, as the degree of symmetry, the difference in volume or area (or the ratio) between the right and left cerebral hemispheres may be adopted. Where the difference in volume or area or the ratio is in excess of a given threshold value, the volume or area is displayed in a color different from colors used where the difference or ratio is less than its threshold value, together with the difference or ratio. In this way, the presence of swelling or shrinkage is emphasized.

The incipient slice position of the image displayed on the display screen of FIG. 24 is determined as follows. The control portion 41 detects a slice where a basal ganglia that is clinically valuable is depicted, using any one of the following slices or any combination of them.
(a) A slice located at 2 cm over a slice that makes an upper part of the ear invisible
(b) Slices showing cerebral ventricles that are separated into four. These slices have sizes greater than a certain size.
(c) Slices showing the fissura Sylvia that is seen to have a certain degree of size.

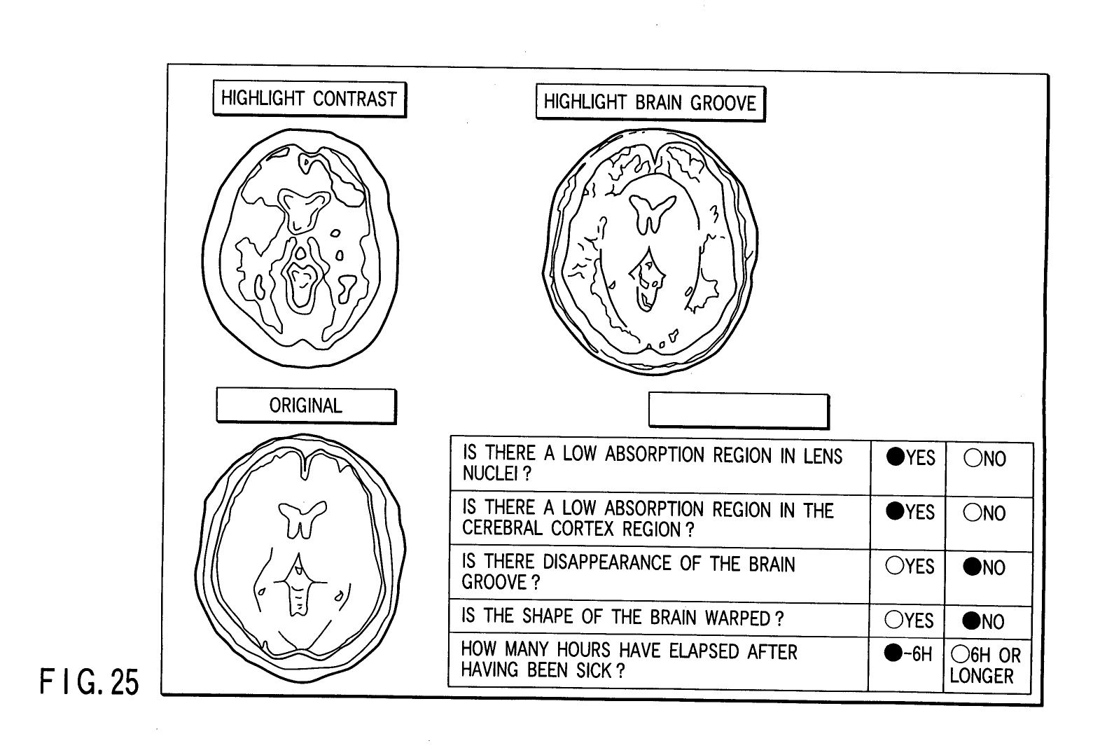

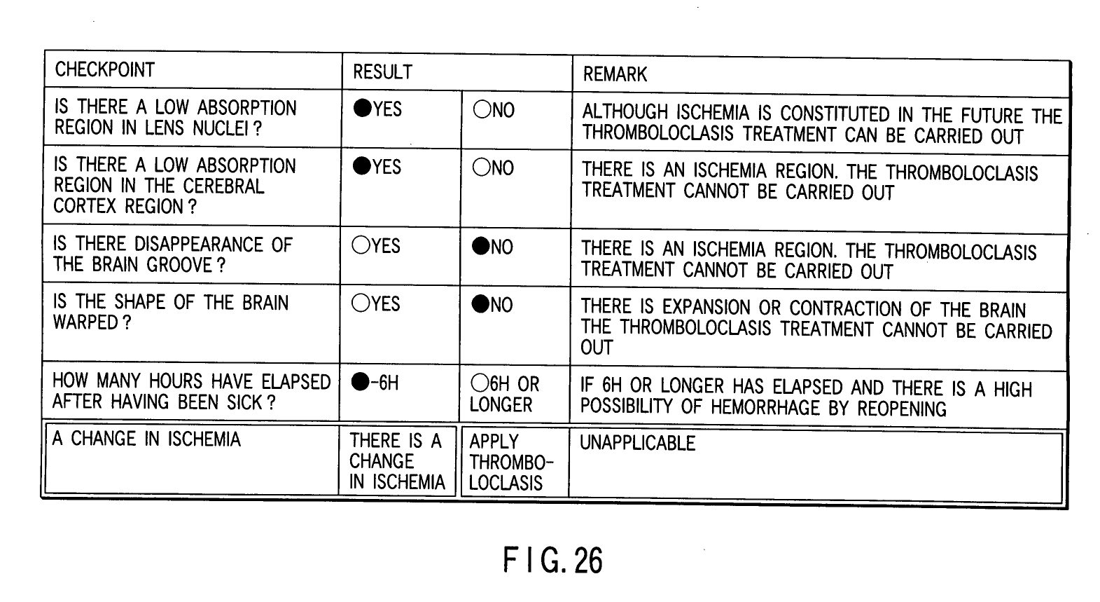

Display of diagnostic guidelines provided by the guideline generating portion 45 is next described. For example, in the viewing screen of FIG. 24, a button having a message “Guide on Diagnosis and Therapy” is displayed. By clicking this button, guidelines on diagnosis of cerebral ischemia are displayed as shown in FIGS. 25 and 26. Plural items to be checked by the user for diagnosis of cerebral ischemia are prepared in the guidelines, together with remarks. Examples of the items are given below.
“Is there a low absorption region in the lenticular nucleus?” (Remark: An ischemia will occur in future but a clot-dissolving treatment is still applicable.)
“Is there a low absorption region in cortex?” (Remark: There is an ischemic region. A clot-dissolving treatment is inapplicable.)
“Is there effacement of cerebral sulci?” (Remark: There is an ischemic region. A clot-dissolving treatment is inapplicable.)
“Is the brain distorted?” (Remark: the brain swells or shrinks. A clot-dissolving treatment is inapplicable.)
“How many hours has it been since the onset?” (Remark: If more than 6 H have passed, bleeding is likely to occur on reopening.)

The guideline generating portion 45 stores presence or absence of variation of ischemia (result of diagnosis of ischemia) and the advisability (precautions taken in the treatment) of the application of a treatment (e.g., blood clot-dissolving treatment) are stored in a corresponding manner to combinations of answers to these checked items. The presence or absence of variation of ischemia and advisability of application of the treatment (blood clot-dissolving treatment) for the corresponding combinations of answers to the checked items are displayed.

A diagnosis using cerebral blood flow (CBP study) is next described. As shown in FIG. 13, the CBP study processing portion 120 is made up of an ROI setting aiding portion 121, a time concentration curve creating portion 122, a cerebral artery time concentration curve correcting portion 123, an MTF processing portion 124, an index calculating portion 125, a map creating portion 126, and a map synthesizing portion 127. The CBP study is described briefly. The CBP study is a procedure for calculating indexes regarding the dynamic state of blood flow through blood capillaries within cerebral tissues. In the CBP study, indexes such as CBP, CBV, MTT, and Err quantitatively expressing the dynamic state of a local blood flow in a tissue, i.e., the dynamic state of blood flow passing through the blood capillaries within a local tissue, are found. Also, maps of these indexes are output.

CBP indicates the flow rate of blood [ml/100 ml/min] per unit volume and per unit time within blood capillaries in a cerebral tissue. CBV indicates the amount of blood [ml/100 ml] per unit volume within a cerebral tissue. MTT indicates the mean blood transit time [seconds] through blood capillaries. Err indicates the total sum of residual errors or the square root of the sum of squares of residual errors when a transfer function is approximated.

The indexes CBP, CBV, and MTT that quantitatively express the dynamic state of blood flow in the blood capillaries within a cerebral tissue are expected to be information useful for identification of the sick body of an ischemic cerebrovascular disease, evaluation as to whether blood capillaries have enlarged or not, and evaluation of blood velocity, together with information about the elapsed time from the onset of a cerebral ischemic stroke. For example, in an ischemic cerebrovascular disorder, the blood pressure of the feeding artery generally drops. It is observed that the blood flow velocity within this blood vessel drops. As a result, even if CBV is constant, MTT prolongs and CBP drops. In addition, in the superacute phase of cerebral infarction, the blood capillaries are expanded and the blood flow velocity is increased to compensate for the drop of the blood flow velocity due to a blood pressure drop. This suppresses drop of the blood flow rate CBP. This action is known as autoregulation. Accordingly, where MTT is prolonged, if CBP drops but CBV increases, then information suggesting the possibility that the blood capillaries have reopened is available.

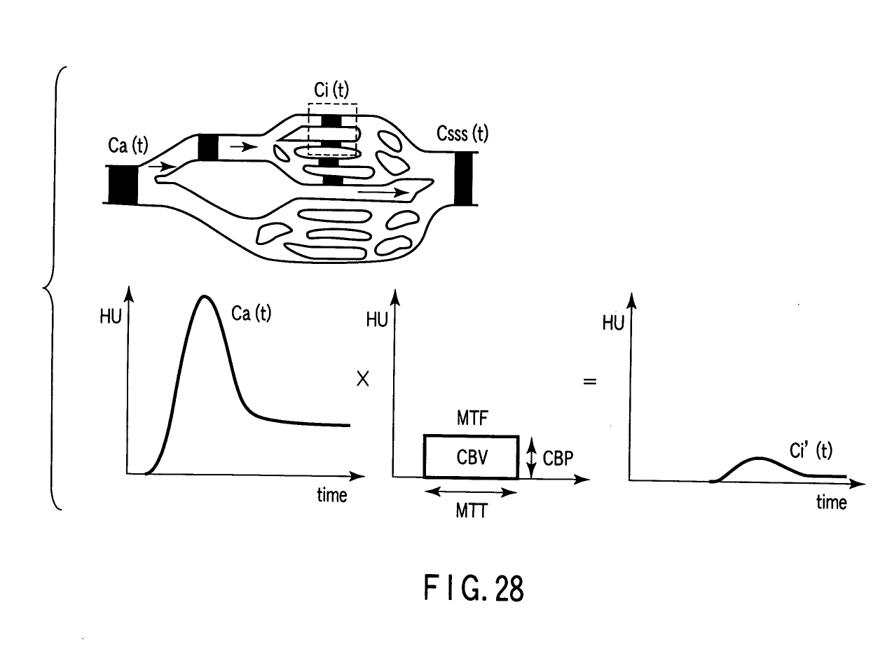

In the CBP study, a contrast medium that does not penetrate through cerebral blood vessels such as an iodinated contrast medium is used as a tracer. The iodinated contrast medium is injected from an elbow vein by an injector, for example. The iodinated contrast medium intravenously injected by the injector flows into the cerebral arteries through the heart and lungs. Then, the medium passes through the blood capillaries within the cerebral tissues from the cerebral arteries. Then, the medium flows out into the cerebral veins. At this time, the iodinated contrast medium passes through the blood capillaries within cerebral tissues without leaking out from the capillaries if the cerebral tissues are normal. This state is schematically shown in FIG. 28.

The manner in which the contrast medium passes is imaged by dynamic CT. From the resulting successive images, time concentration curve Ca(t) of pixels on the cerebral-arteries, time concentration curve Ci(t) of pixels on cerebral tissues (blood capillaries), and time concentration curve Csss(t) of pixels on cerebral veins are measured.

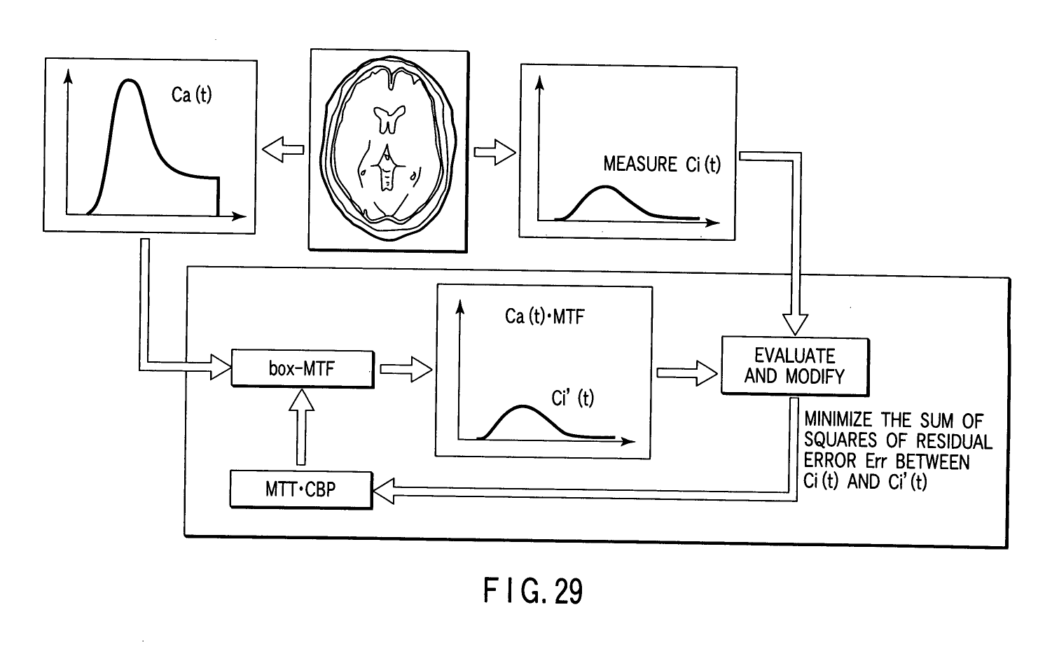

In this CBP study, as shown in FIG. 29, an ideal relation holding between the concentration time curve Ca(t) of the cerebral arteries and the concentration time curve Ci(t) of the cerebral tissues is used as an analysis model. Where the contrast medium is injected from a blood vessel about to enter cerebral tissues, the rising edge of the time concentration curve in a unit volume (1 pixel) in the cerebral tissues is vertical and maintained at a constant value for a while. Then, the curve falls steeply. This is approximated by a rectangular function (box-MTF (modulation transfer function) method).

That is, the transfer function between the input function and the output function is approximated by a rectangular function under the conditions where the time concentration curve Ca(t) of the cerebral arteries is taken as the input function and the time concentration curve Ci(t) of cerebral tissues is taken as the output function. The transfer function indicates the process of the tracer passing through blood capillaries.

The time concentration curve creating portion 122 creates a time concentration curve about the cerebral arteries, cerebral veins, and cerebral tissues (blood capillaries) from data about dynamic CT images (data about images that are successive in terms of time) stored in the storage device 10M. The time concentration curve Ca(t) about the cerebral arteries is created for each of the set 6 cerebral artery ROIs, for example. The time concentration curve Csss(t) about the cerebral veins is created regarding the cerebral vein ROI set on a superior longitudinal sinus. Furthermore, the time concentration curve Ci(t) about cerebral tissues are created for each of all the pixels on cerebral tissues.

To remove the influences of the effects of noises and partial volume effects, the cerebral artery time concentration curve correcting portion 123 corrects the time concentration curve Ca(t) about the cerebral arteries based on the time concentration curve Csss(t) about superior longitudinal sinuses. This method of correction will be described later. The MTF processing portion 124 calculates the transfer function MTF for, each of all the pixels within the cerebral tissue regions by the box-MTF method based on the corrected time concentration curve Ca(t) about the cerebral arteries and on the time concentration curve Ci(t) about the cerebral tissues.

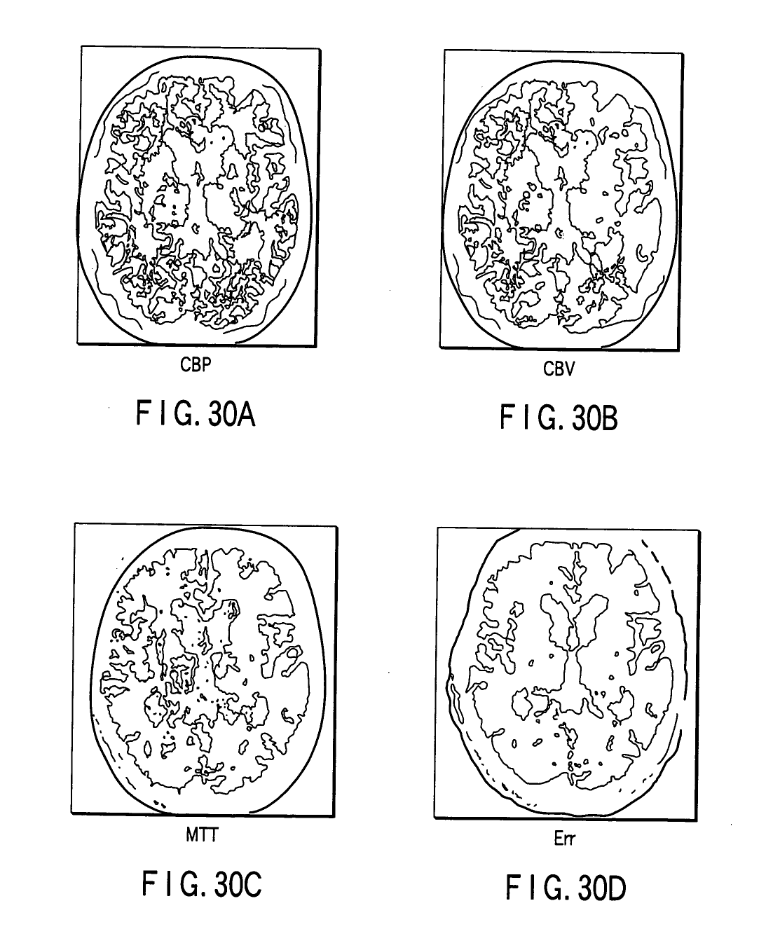

The index calculating portion 125 calculates the indexes (CBP, CBV, MTT, and Err) indicating the dynamic state of the blood flow through the cerebral tissues from the calculated transfer function MTF for each of all the pixels within the cerebral tissue areas. The map creating portion 126 creates maps of the calculated indexes for each of cerebral arteries (ACA, MCA, and PCA) as shown in FIG. 30.

As shown in FIG. 27, by combining a diagnosis using CT value images and a diagnosis using cerebral blood flow images, a diagnosis with accuracy higher than that of the diagnosis using only one of these two kinds of images and its treatment policy can be determined. Especially, low CT value regions from CT value images can be estimated as irrecoverable regions, which are contained within the low blood flow regions. The region that is within the low blood flow regions and outside the irrecoverable regions can be regarded as highly recoverable regions. The size of these regions is used as an important factor in making a judgment as to treatment effects. In the embodiment make the gray matter as obvious as possible. These metabolically active areas infarction first. If they are abnormal on an un-enhanced head CT they are infracted, but they are not easy for everyone to see and we are creating a means of making the infarction detection easier.

### THIRD EMBODIMENT

An apparatus for aiding the diagnosis and treatment of acute cerebral infarction, according to the third embodiment of the invention, will be described with reference to the drawings. The apparatus according to this embodiment uses a three-dimensional CT image that an X-ray CT scanner has acquired. The three-dimensional CT image is of either multi-slice type or volume type that is a set of voxels. Here, the CT image will be described as a multi-slice type one.

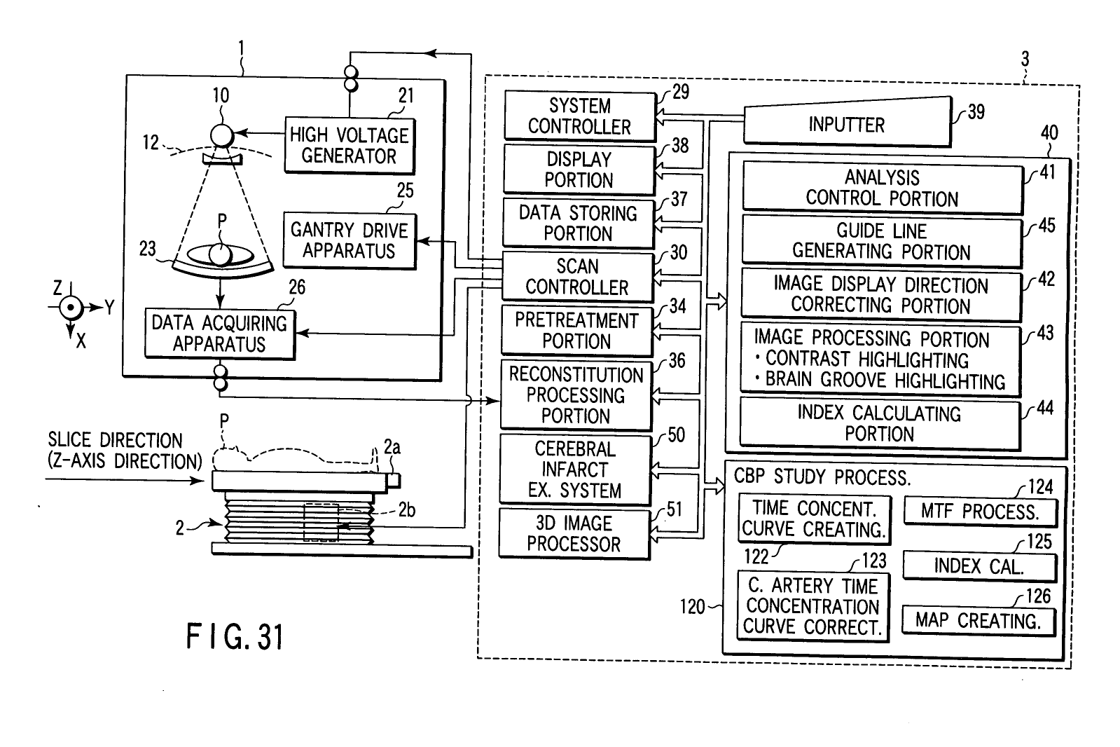

As seen from FIG. 31, the X-ray CT scanner used in the present embodiment is similar to its counterpart of the second-embodiment. It differs in two respects. First, the image processing portion 43 can extract images of the blood vessels. Second, the apparatus further comprises a 3D-image processing portion 51.

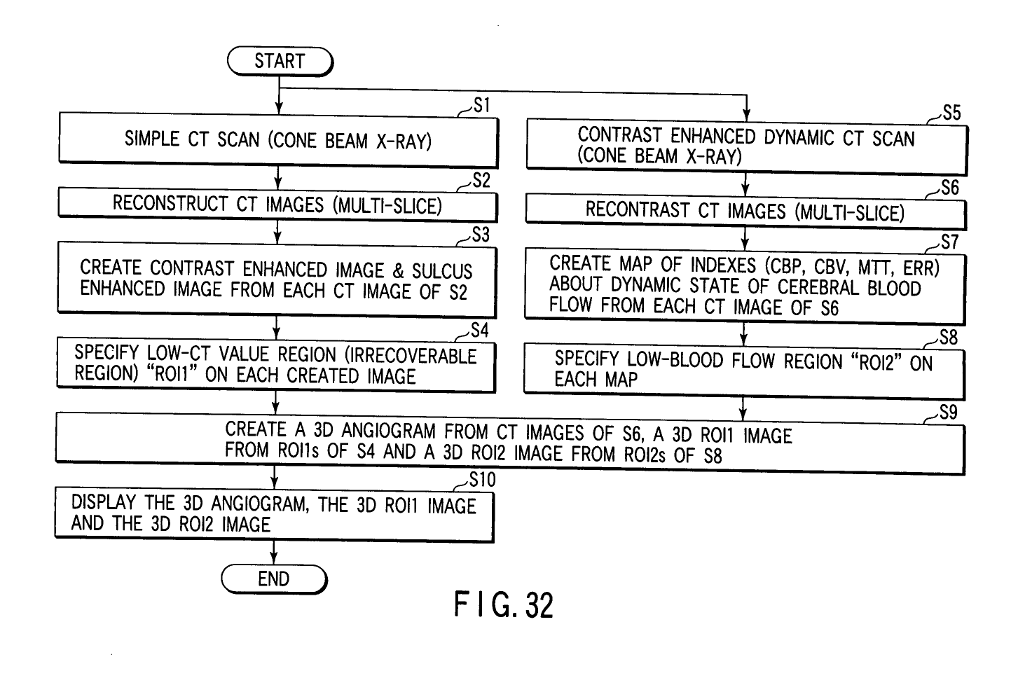

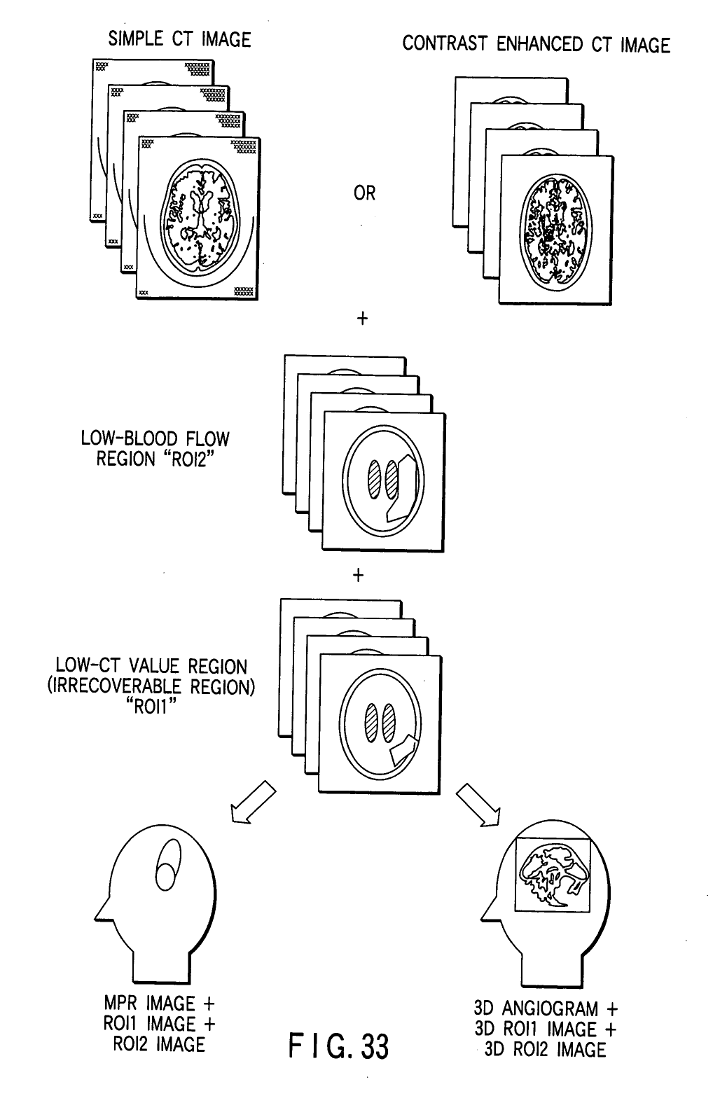

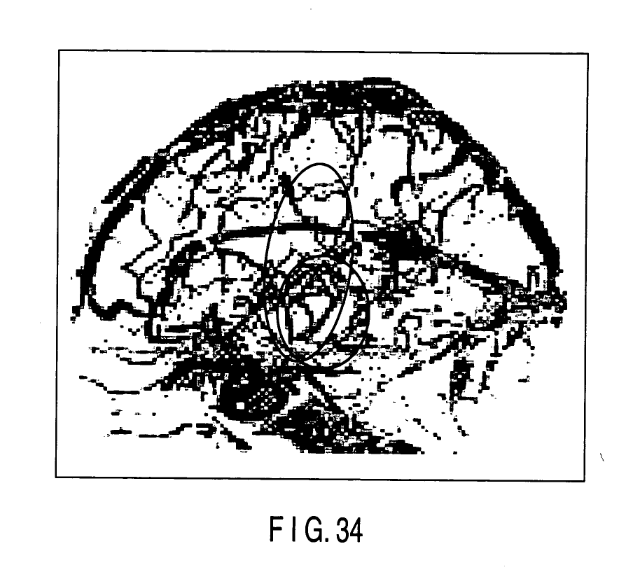

As FIGS. 32 and 33 show, simple CT scanning is performed, acquiring projection data representing a 3-dimensional region of the patient's head (S1). Contrast dynamic CT scanning is also performed in step S5, thereby acquiring projection data representing the same three-dimensional region. The contrast dynamic CT scanning is typically volume scanning that uses cone-beam X rays. As described above, the simple CT scanning uses no contrast media, and the contrast dynamic CT scanning uses a contrast medium.

Using the projection data acquired by the simple CT scanning (S1), the reconstruction processing portion 36 reconstructs CT image data for a plurality of slices (S2). The CT image is processed for each slice, thus generating a contrast-enhanced image and a cerebral sulci-enhanced image (S3). The display portion 38 selectively displays the contrast-enhanced image, along with the cerebral sulci-enhanced image. The operator operates the input device 39, displaying a frame on the contrast-enhanced image or cerebral sulci-enhanced image. A low-CT value region ((first ROI) (i.e., irrecoverable region)) is thereby specified. A first ROI is specified for one slice (S4).

Using the projection data acquired in the contrast dynamic CT scanning (S5), the reconstruction processing portion 36 reconstructs CT images, each for one slice (S6). The CBP study processing portion 120 generates maps of CBP, CBV, MTT, Err indices, respectively, for each slice (S7). The display portion 38 displays the map of CBP, together with the map of CBV, MTT or Err.

The operator operates the input device 39, displaying a frame (second ROI) on, for example, the map of CBP, thus specifying a low blood flow region for one slice.

Next, the image processing portion 43 extracts images of blood vessels, each for one slice, from the CT image reconstructed in step S6. Since the contrast medium has been applied to blood vessels, the portion 43 extracts the images of the blood vessels, at high precision. A 3D-angiogram is generated for any slice, from the image of blood vessels (S9). The position of the blood vessel with the anomaly from the image of 3D-angiogram can be confirmed. Likewise, two 3D-images of the first and second ROIs designated for the slice, respectively, are generated. The 3D-image processing portion 51 superimposes the 3D-images of the first and second ROIs on the angiogram, forming a combined image, and the display portion 38 displays the combined image (S10).

The 3D-image processing portion 51 performs multiplanar reconstruction processing (MPR), thus generating a CT image for the plane designated by the operator, from CT images of a plurality of slices. Similarly, the 3D-image processing portion 51 generates two images of the first and second ROIs designated for each slice. The 3D-image processing portion 51 superimposes the images of the first and second ROIs on the CT image, forming a combined image. The display portion 38 displays the combined image thus formed.

The third embodiment of the invention can provide a three-dimensional image, which helps the observer to understand the positional relation of the blood vessel with the anomaly, the irrecoverable region and the low blood flow region.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A cerebral ischemia diagnosis assisting apparatus comprising:
a storing portion for storing multi-slices or volume data with regard to the head portion of a subject;
an image generating portion for generating a tomography image of the brain from the multi-slices or the volume data;
an image processing portion for processing the tomography image for generating a contrast highlighting image and a brain sulci highlighting image or either thereof; and
a display portion for displaying the tomography image along with the contrast highlighting image and the brain sulci highlighting image.

2. The cerebral ischemia diagnosis assisting apparatus according to claim 1, wherein the image generating portion generates the tomography image of a face orthogonal to a central face separating the left and right cerebral hemisphere from the multi-slices or the volume data.

3. The cerebral ischemia diagnosis assisting apparatus according to claim 1, wherein the image generating portion detects a plurality of center lines for separating the left and right cerebral hemisphere from a plurality of the tomography images initially generated from the multi-slices or the volume data for generating the central face to minimize an error of a positional shift relative to the plurality of center lines.

4. The cerebral ischemia diagnosis assisting apparatus according to claim 1, wherein the image processing portion generates the contrast highlighting image by classifying a brain tissue region of the tomography image at least by three kinds of colors in accordance with a CT value.

5. The cerebral ischemia diagnosis assisting apparatus according to claim 1, wherein the image processing portion generates the brain sulci highlighting image by classifying a cerebral spinal fluid region of the tomography image at least by two kinds of colors in accordance with a CT value.

6. The cerebral ischemia diagnosis assisting apparatus according to claim 1, further comprising a calculating portion for calculating a ratio between volumes of a left cerebral hemisphere region and a right cerebral hemisphere region separated by the central face.

7. The cerebral ischemia diagnosis assisting apparatus according to claim 1, further comprising a calculating portion for calculating a ratio between areas of a left cerebral hemisphere region and a right cerebral hemisphere region of the tomography image.

8. The cerebral ischemia diagnosis assisting apparatus according to claim 1, further comprising a calculating portion for calculating a correlation coefficient between the tomography image and a left and right inverted image of the tomography image.

9. The cerebral ischemia diagnosis assisting apparatus according to claim 1, further comprising a determining portion for determining applicability of a treatment based on a combination of a plurality of diagnosis results by a determination of a user.

10. An X-ray computer tomography apparatus which is an X-ray computer tomography apparatus for reconstituting multi-slices or volume data with regard to the head portion of a subject based on data acquired by scanning the subject, said X-ray computer tomography comprising:
an image generating portion for generating a tomography image of the brain from the multi-slices or the volume data;
an image processing portion for processing the tomography image for generating a contrast highlighting image and a brain sulci highlighting image; and
a display portion for displaying the tomography image along with the contrast highlighting image and the brain sulci highlighting image.

11. The cerebral ischemia diagnosis assisting apparatus according to claim 10, wherein the image generating portion generates a tomography image of a face orthogonal to a central face separating the left and right cerebral hemisphere from the multi-slices or the volume data.

12. An apparatus for aiding diagnosis and treatment of acute cerebral infarction, comprising:
a storage portion for storing CT images regarding cross sections of the head of a patient to be examined and cerebral blood flow images indicating a spatial distribution of indexes indicating dynamic state of cerebral blood flow regarding cross sections of said patient substantially identical to the first-mentioned cross sections;
an image processing portion for superimposing a first ROI regarding ischemia identified from said CT images and a second ROI regarding low blood flow identified from said cerebral blood flow images onto said CT images; and
a display portion for displaying said CT images on which said first ROI and said second ROI have been superimposed.

13. An apparatus for aiding diagnosis and treatment of acute cerebral infarction according to claim 12, further comprising an image creating portion for creating at least one kind of contrast-enhanced images created from said CT images by emphasizing regions having CT values lying in a certain range of CT values and cerebral sulci-enhanced images created from said CT images by emphasizing cerebral sulci regions.

14. An apparatus for aiding diagnosis and treatment of acute cerebral infarction according to claim 12, further comprising an image creating portion for calculating and displaying a blood flow rate per unit volume and per unit time within blood capillaries of cerebral tissues and an amount of blood per unit volume within cerebral tissues or a mean blood transit time through blood capillaries as said indexes indicating the dynamic state of the cerebral blood flow.

15. An apparatus for aiding diagnosis and treatment of acute cerebral infarction according to claim 12, further comprising a calculating portion for calculating the ratio of the area of the difference between said second ROI and said first ROI to said second ROI.

16. An apparatus for aiding diagnosis and treatment of acute cerebral infarction according to claim 12, wherein a differential region between said second ROI and said first ROI is displayed with emphasis in a color different from a color used for displaying said second ROI.

17. An apparatus for aiding diagnosis and treatment of acute cerebral infarction according to claim 12, further comprising an input device for entering said first and second ROIs.

18. An apparatus for aiding the diagnosis and treatment of acute cerebral infarction, comprising:
a storage portion for storing a three-dimensional CT image of the head of a patient and a three-dimensional contrast CT image of the head of the patient;
an image processing portion for superimposing a three-dimensional image of a first ROI in the infract region identified from the CT image and a three-dimensional image of a second ROI in a low-blood flow region identified from the contrast CT image, onto a blood vessel image generated from the contrast CT image; and
a display portion for displaying the image of the blood vessels, which is superimposed with the three-dimensional images of the first and second ROIs.

19. The apparatus according to claim 18, wherein a two-dimensional CT image of a given slice is generated from the three-dimensional CT image, a two-dimensional image of the first ROI is generated from the three-dimensional image of the first ROI, a two-dimensional image of the second ROI is generated from the three-dimensional image of the second ROI, and the two-dimensional images of the first and second ROIs are superimposed on the two-dimensional CT image.
